(19) United States
(12) Patent Application Publication
(10) Pub. No.: US 2002/0178512 A1
(43) Pub. Date: Dec. 5, 2002

(54) Oxidation dyeing composition for keratinous fibres comprising a n-(2-hydrobenzene)-carbamate or a n-(2-hydroxy-benzene)-urea and a pyrazolopyrimine, dyeing methods

(76) Inventors: Florent Pastore, Malmaison (FR); Sylvain Kravtchenko, Asnieres (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/959,247

(22) PCT Filed: Feb. 21, 2001
(86) PCT No.: PCT/FI01/00502

(30) Foreign Application Priority Data
Feb. 23, 2000 (FR) 00/02336

(51) Int. Cl. A61K 7/13

(52) U.S. Cl. 8/405; 8/406; 8/408

(57) ABSTRACT

The subject of the invention is a composition for the oxidation dyeing of keratinous fibers, and in particular human keratinous fibers such as hair, comprising at least one oxidation base, chosen from the pyrazolopyrimidines, and, as coupler, at least one N-(2-hydroxybenzene)carbamate or one N-(2-hydroxy-benzene)urea of formula (I), their use as coupler for the oxidation dyeing of keratinous fibers, and the oxidation dyeing methods using them.

The subject of the invention is a composition for the oxidation dyeing of keratinous fibres, and in particular human keratinous fibres such as hair, comprising at least one oxidation base, chosen from the pyrazolopyrimidines, and, as coupler, at least one N-(2-hydroxybenzene)carbamate or one N-(2-hydroxy-benzene)urea of formula (I), their use as coupler for the oxidation dyeing of keratinous fibres, and the oxidation dyeing methods using them.

It is known to dye keratinous fibres and in particular human hair with dyeing compositions containing oxidation dye precursors, in particular para-phenylenediamines, ortho- or para-aminophenols, heterocyclic compounds such as diaminopyrazole derivatives, generally called oxidation bases. Oxidation dye precursors, or oxidation bases, are colourless or weakly coloured compounds which, combined with oxidizing products, can give rise, through a process of oxidative condensation, to coloured or colouring compounds.

It is also known that it is possible to vary the shades obtained with these oxidation bases by combining them with couplers or colour modifiers, the latter being chosen in particular from aromatic meta-diamines, meta-aminophenols, meta-diphenols, noncationic naphthols or alternatively certain heterocyclic compounds such as for example indolic couplers.

The variety of molecules used in oxidation bases and couplers allows a rich palette of colours to be obtained.

The so-called <<permanent>> colour obtained using these oxidation dyes must moreover satisfy a number of requirements. Thus, it must be without drawbacks from the toxicological point of view, it must make it possible to obtain shades in the desired intensity and exhibit good resistance towards external agents (light, adverse weather conditions, washing, permanent waving, perspiration, rubbing).

The dyes must also make it possible to cover grey hair and must finally be the least selective possible, that is to say make it possible to obtain the smallest possible differences in colour right along the same keratinous fibre, which may indeed be differently sensitized (i.e. damaged) between its tip and its root.

Oxidation dyeing compositions containing certain derivatives of N-(2-hydroxy-4-aminophenyl)-acetamide as couplers have already been proposed, in particular in patent applications WO 98/52519, JP2521636, FR-A-1 596 879 or FR-A-2 233 984, in combination with oxidation bases conventionally used in oxidation dyeing such as, for example, para-phenylenediamines, para-aminophenols or even some pyridines. However, such compositions are not always satisfactory, in particular from the point of view of the intensity and the chromaticity of the colours obtained.

However, the applicant has now just discovered, completely unexpectedly and surprisingly, that it is possible to obtain new dyes which can lead to intense colours which are particularly chromatic and brilliant, which are not very selective, and which exhibit excellent properties of resistance to the various attacks to which keratinous fibres may be subjected by combining at least one oxidation base chosen from pyrazolopyrimidines and at least one coupler chosen from N-(2-hydroxybenzene)carbamates and N-(2-hydroxybenzene)ureas of formula (I) defined below.

These discoveries form the basis of the present invention.

The first subject of the invention is therefore a composition for the oxidation dyeing of keratinous fibres, and in particular human keratinous fibres such as hair, characterized in that it contains, in a medium appropriate for dyeing:

at least one oxidation base chosen from the pyrazolopyrimidines;

and at least one coupler chosen from the compounds of the following formula (I), and their addition salts with an acid:

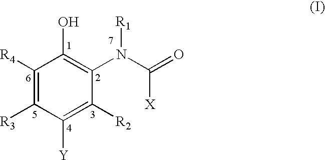

in which:

R₁ represents a hydrogen atom; a radical comprising from 1 to 15 carbon atoms, linear or branched (it being possible for the branch(es) to form one or more carbon-containing rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; it being understood that the said SO₂ group is not directly linked to the nitrogen atom at the 7-position carrying the radical R₁; the said radical R₁ not comprising a peroxide bond or diazo, nitro or nitroso radicals;

X represents a group OR₅ or NR₆R₇;

R₅ represents a radical comprising from 1 to 20 carbon atoms, linear or branched (it being possible for the branch(es) to form one or more carbon-containing rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R₅ not comprising a peroxide bond or diazo, nitro or nitroso radicals;

R₆ and R₇, which are identical or different, represent a hydrogen atom; a radical comprising from 1 to 20 carbon atoms, linear or branched (it being possible for the branch(es) to then form one or more rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radicals R₆ and R₇ not comprising a peroxide bond or diazo, nitro or nitroso radicals;

the radicals R₆ and R₇ may, in addition, be linked in order to form a saturated or unsaturated ring comprising from 5 to 7 members, consisting of carbon, nitrogen, oxygen, sulphur and/or the group C═O, each member being unsubstituted or substituted with 1 or 2 radicals R, which are identical or different, R being a C₁-C₆ alkyl radical, linear or branched (it being possible for the branch(es) to then form one or more rings comprising from 3 to 6 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R not comprising a peroxide bond or diazo, nitro or nitroso radicals;

the radicals R₁ and R₅ may, in addition, be linked in order to form a saturated or unsaturated ring comprising from 5 to 7 members, consisting of carbon, nitrogen, oxygen, sulphur and/or the group C═O, each member being unsubstituted or substituted with 1 or 2 radicals R, which are identical or different, R being a C₁-C₆ alkyl radical, linear or branched (it being possible for the branch(es) to then form one or more rings comprising from 3 to 6 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R not comprising a peroxide bond or diazo, nitro or nitroso radicals;

the radicals R₁ and R₆ may, in addition, be linked in order to form a saturated or unsaturated ring comprising from 5 to 7 members, consisting of carbon, nitrogen, oxygen, sulphur and/or the group C═O, each member being unsubstituted or substituted with 1 or 2 radicals R, which are identical or different, R being a C₁-C₆ alkyl radical, linear or branched (it being possible for the branch(es) to then form one or more rings comprising from 3 to 6 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R not comprising a peroxide bond or diazo, nitro or nitroso radicals;

R₂, R₃ and R₄, which are identical or different, represent a hydrogen or halogen atom; a radical comprising from 1 to 20 carbon atoms, linear or branched (it being possible for the branch(es) to then form one or more rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radicals R₂, R₃ and R₄ not comprising a peroxide bond or diazo, nitro or nitroso radicals; it being understood that R₄ cannot represent a hydroxyl, thio or amino radical or a sulphonylamino group which is substituted or unsubstituted; and it being understood that the radicals R₂, R₃ and R₄ cannot be linked to the benzene ring in formula (I) by an —NH—NH— bond; the radicals R₁ and R₂ may, in addition, be linked so as to form a saturated ring comprising from 5 to 7 members, consisting of carbon, nitrogen, oxygen, sulphur and/or the group C═O, each member being unsubstituted or substituted with 1 or 2 radicals R, which are identical or different, R having the same meanings as those indicated above; the said radical R not comprising a peroxide bond or diazo, nitro or nitroso radicals;

the radicals R₂ and R₅ may also be linked so as to form a saturated ring comprising from 5 to 7 members, consisting of carbon, nitrogen, oxygen, sulphur and/or the group C═O, each member being unsubstituted or substituted with 1 or 2 radicals R, which are identical or different, R having the same meanings as those indicated above; the said radical R not comprising a peroxide bond or diazo, nitro or nitroso radicals;

the radicals R₂ and R₆ may also be linked so as to form a saturated ring comprising from 5 to 7 members, consisting of carbon, nitrogen, oxygen, sulphur and/or the group C═O, each member being unsubstituted or substituted with 1 or 2 radicals R, which are identical or different, R having the same meanings as those indicated above; the said radical R not comprising a peroxide bond or diazo, nitro or nitroso radicals;

Y represents a hydrogen or halogen atom; a group —OR₈, —SR₈, or —NH—SO₂R₈ in which R₈ represents a C₁-C₈ alkyl radical, linear or branched (it being possible for the branch(es) to then form one or more rings comprising from 3 to 6 members), unsubstituted or substituted with one or more radicals chosen from the group consisting of a halogen atom, a hydroxyl, C₁-C₄ alkoxy, amino and amino(C₁-C₄ alkyl) radical; a phenyl radical, unsubstituted or substituted with one or two radicals chosen from the group consisting of a C₁-C₄ alkyl, trifluoromethyl, carboxyl, C₁-C₄ alkoxycarbonyl, halogen, hydroxyl, C₁-C₄ alkoxy, amino and amino(C₁-C₄ alkyl) radical; or a benzyl radical; it being understood that when R₂ represents a hydroxyl radical, then Y cannot represent a group —NH—SO₂R₈;

it being understood that:

when R₂ and R₄ simultaneously represent a hydrogen atom and R₃ represents a substituted or unsubstituted C₁-C₄ alkyl radical, then X cannot represent a group OR₅ in which R₅ represents a substituted or unsubstituted C₁-C₄ alkyl radical;

when R₂ and R₄ simultaneously represent a hydrogen atom and R₃ represents a substituted or unsubstituted CL-C₄ alkyl radical, then X cannot represent a group NR₆R₇;

when R₂, R₄ and Y simultaneously represent a hydrogen atom, and X represents a group NR₆R₇ in which R₆ and R₇ simultaneously represent a hydrogen atom, then R₃ is different from a fluorine, chlorine or bromine atom;

when X represents a group NR₆R₇ and R₁ and R₆, which are identical or different, represent a hydrogen atom or a C₁-C₄ alkyl radical or a group —CH₂CONH₂ or —CH₂CON(CH₂CH₃)₂ and R₂ represents a hydrogen atom or a C₁-C₄ alkoxy radical, and R₄ represents an amino radical which is mono- or disubstituted with a C₁-C₄ alkyl or an aryl radical, and R₇ represents a hydrogen atom or a C₁-C₄ alkyl radical or a substituted or unsubstituted aromatic group or a substituted or unsubstituted heteroaromatic group and Y represents a hydrogen or halogen atom or a C₁-C₄ alkoxy group, then R₃ is different from a hydrogen or halogen atom, and from a hydroxyl, amino, C₁-C₄ alkyl, C₁-C₄ alkoxy or aryl group;

when X represents a group NR₆R₇ and R₁ and R₆, which are identical or different, represent a hydrogen atom, a C₁-C₄ alkyl or a group —CH₂CONH₂ or —CH₂CON(CH₂CH₃)₂, and R₄ represents a hydrogen or halogen atom or a C₁-C₄ alkyl, C₁-C₄ alkoxy, or aryl radical, and R₇ represents a hydrogen atom, a C₁-C₄ alkyl radical or a substituted or unsubstituted aromatic group, or a substituted or unsubstituted heteroaromatic group and Y represents a C₁-C₄ alkoxy radical, then R₂ and R₃ are different from a hydrogen or halogen atom, and from a hydroxyl, amino, C₁-C₄ alkyl, C₁-C₄ alkoxy or aryl group.

As indicated above, the colours obtained with the oxidation dyeing composition in accordance with the invention are intense and particularly brilliant and chromatic and make it possible to obtain, in particular, red shades containing no or very little blue or yellow. They furthermore exhibit excellent properties of resistance towards the action of various external agents (light, adverse weather conditions, washing, permanent waving, perspiration, rubbing).

The subject of the invention is also a method for the oxidation dyeing of keratinous fibres using this dyeing composition.

Among the pyrazolopyrimidines which may be used as oxidation base in the dyeing composition in accordance with the invention, there may be mentioned in particular those mentioned in patent applications FR-A-2 750 048 and FR-A-2 771 631 whose contents form an integral part of the application and among which there may be mentioned pyrazolo[1,5-a]pyrimidine-3,7-diamine; 2-methylpyrazolo[1,5-a]pyrimidine-3,7-diamine; 5-methylpyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)-(2-hydroxyethyl)amino]ethanol; 2-[(7-aminopyrazolo-[1,5-a]pyrimidin-3-yl)-(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine; 3-amino-5-methyl-7-imidazolylpropylamino-pyrazolo[1,5-a]pyrimidine, 3-amino-5,6-dimethyl-7-imidazolylpropylaminopyrazolo[1,5-a]pyrimidine; pyrazolo[1,5-a]pyrimidin-3-ylamine; 5,7-dimethylpyrazolo[1,5-a]pyrimidin-3-ylamine; 5,6,7-trimethylpyrazolo[1,5-a]pyrimidin-3-ylamine; 7-methylpyrazolo[1,5-a]pyrimidin-3-ylamine; 5-methylpyrazolo[1,5-a]pyrimidin-3-ylamine; 7-methoxypyrazolo[1,5-a]pyrimidin-3-ylamine; 5-methoxypyrazolo[1,5-a]pyrimidin-3-ylamine; 7-methoxy-5-methylpyrazolo[1,5-a]pyrimidin-3-ylamine; 2,5,6,7-tetramethylpyrazolo[1,5-a]pyrimidin-3-ylamine; 2-methoxy-5,7-dimethylpyrazolo[1,5-a]pyrimidin-3-ylamine; 5,7-di-tert-butylpyrazolo[1,5-a]pyrimidin-3-ylamine; 5,7-di-trifluoromethylpyrazolo-[1,5-a]pyrimidin-3-ylamine; 2,6-dimethylpyrazolo-[1,5-a]pyrimidin-3-ylamine; 2-chloro-5,7-dimethylpyrazolo[1,5-a]pyrimidin-3-ylamine; 7-methyl-2-tert-butyl-5-trifluoromethylpyrazolo[1,5-a]pyrimidin-3-ylamine; 3-amino-7-imidazolylpropylaminopyrazolo-[1,5-a]pyrimidine; 3-amino-7-dimethylaminoethylaminopyrazolo[1,5-a]pyrimidine; 7-methoxy-2,5-dimethylpyrazolo[1,5-a]pyrimidin-3-ylamino; 7-methoxy-2-methylpyrazolo[1,5-a]pyrimidine-3,5-diamine; 7-methoxy-2-methylpyrazolo[1,5-a]pyrimidin-3-ylamine; 7-methoxy-2,5,6-trimethylpyrazolo[1,5-a]pyrimidin-3-ylamine; 7-methoxy-2,6-dimethylpyrazolo[1,5-a]-pyrimidine-3,5-diamine; 7-methoxy-2,6-dimethylpyrazolo-[1,5-a]pyrimidin-3-ylamine; 2-(3-amino-2,5-dimethylpyrazolo[1,5-a]pyrimidin-7-yloxy)ethanol; 2-(3,5-diamino-2-methylpyrazolo[1,5-a]pyrimidin-7-yloxy)ethanol; 2-(3-amino-2-methylpyrazolo[1,5-a]-pyrimidin-7-yloxy)ethanol; 2-(3-amino-2,5,6-trimethylpyrazolo[1,5-a]pyrimidin-7-yloxy)ethanol; 2-(3,5-diamino-2,6-dimethylpyrazolo[1,5-a]pyrimidin-7-yloxy)ethanol; 2-(3-amino-2,6-dimethylpyrazolo-[1,5-a]pyrimidin-7-yloxy)ethanol; 5,6-dimethylpyrazolo[1,5-a]pyrimidin-3-ylamine; 7-methoxy-5,6-dimethylpyrazolo[1,5-a]pyrimidin-3-ylamine; 5,7-dimethylpyrazolo[1,5-a]pyridimin-3-ylamine; pyrazolo[1,5-a]pyrimidine-3,5-diamine; 6-methylpyrazolo[1,5-a]pyrimidine-3,5,7-triamine; 6,7-dimethylpyrazolo[1,5-a]pyrimidine-3,5-diamine; 6-methylpyrazolo[1,5-a]pyrimidine-3,5-diamine; 7-methoxy-6-methylpyrazolo[1,5-a]pyrimidine-3,5-diamine; 3-amino-7-methylpyrazolo[1,5-a]pyrimidin-5-ol; 3,7-diamino-7-methylpyrazolo[1,5-a]pyrimidin-5-ol; 3-amino-6,7-dimethylpyrazolo[1,5-a]pyrimidin-5-ol; 3,7-diamino-6-methylpyrazolo[1,5-a]pyrimidin-5-ol; 2-methoxy-5-methylpyrazolo[1,5-a]pyrimidine-3,7-diamine; 2,7-dimethoxy-5-methylpyrazolo[1,5-a]-pyrimidin-3-ylamine; 2-methoxy-5-methylpyrazolo-[1,5-a]pyrimidin-3-ylamine; 2-methoxypyrazolo[1,5-a]-pyrimidin-3-ylamine; 2-methoxy-7-methylpyrazolo-[1,5-a]pyrimidin-3-ylamine; 2-methoxypyrazolo[1,5-a]-pyrimidine-3,7-diamine; 2,7-dimethoxypyrazolo[1,5-a]-pyrimidin-3-ylamine; and their addition salts with-an acid.

According to a preferred embodiment of the invention, the pyrazolopyrimidines are more particularly chosen from 5-methylpyrazolo[1,5-a]pyrimidine-3,7-diamine; pyrazolo[1,5-a]pyrimidine-3,7-diamine; 2-methylpyrazolo[1,5-a]pyrimidine-3,7-diamine; 3-amino-5-methyl-7-imidazolylpropylaminopyrazolo[1,5-a]-pyrimidine; 3-amino-5,6-dimethyl-7-imidazolylpropylaminopyrazolo[1,5-a]pyrimidine; 2-methoxy-5,7-dimethylpyrazolo[1,5-a]pyrimidin-3-ylamine; 3-amino-7-imidazolylpropylaminopyrazolo-[1,5-a]pyrimidine; and their addition salts with an acid.

The pyrazolopyrimidines preferably represent from 0.0005 to 12% by weight approximately of the total weight of the dyeing composition, and more preferably still from 0.005 to 6% by weight approximately of this weight.

In the compounds of formula (I) in accordance with the invention, when it is indicated that one or more of the carbon atoms of the radical(s) R₁ to R₇ can be replaced with an oxygen, nitrogen or sulphur atom or with an SO₂ group, and/or that the said radicals R₁ to R₇ may contain one or more double bonds and/or one or more triple bonds, this means that it is possible, by way of example, to make the following conversions:

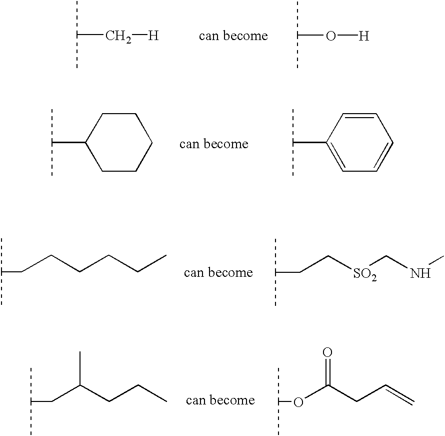

In the compounds of formula (I) according to the invention, R₁ preferably denotes a hydrogen atom; a group A₁, A₂, A₃, A₄ or A₅, the said groups being optionally separated from the nitrogen present at the 7-position to which the radical R₁ is attached by a group —(Co)—.

According to the invention, group A₁ is understood to mean a linear or branched C₁-C₈ alkyl radical which may carry one or two double bonds or one triple bond, which may be unsubstituted or substituted with a group chosen from the groups A₂, A₄ and A₅, which may be unsubstituted or substituted with one or two groups, which are identical or different, chosen from the N-(C₁-C₃)alkylamino, N-(C₁-C₃)alkyl-N-(C₁-C₃)alkylamino, (C₁-C₆)alkoxy, oxo, alkoxycarbonyl, acyloxy, amide, acylamino, ureyl, sulphoxy, sulphonyl, sulphonamido, sulphonylamino, bromo, cyano and carboxyl groups, and may be unsubstituted or substituted with one or more hydroxyl, fluoro or chloro groups.

Group A₂ is understood to mean an aromatic group of the phenyl or naphthyl type, which may be unsubstituted or substituted with one to three groups, which are identical or different, chosen from the methyl, trifluoromethyl, ethyl, isopropyl, butyl, pentyl, fluoro, chloro, bromo, methoxy, trifluoromethoxy, ethoxy, propyloxy, acetyloxy, acetyl and cyano groups.

Group A₃ is understood to mean heteroaromatic groups chosen from the furanyl, thiophenyl, pyrrolyl, imidazolyl, thiazolyl, oxazolyl, 1,2,3-triazolyl, 1,2,4-triazolyl, isoxazolyl, isothiazolyl, pyrazolyl, pyrazolotriazolyl, pyrazoloimidazolyl, pyrrolotriazolyl, pyrazolopyrimidyl, pyrazolopyridyl, pyridyl, pyrimidyl, benzoimidazolyl, benzoxazolyl, benzothiazolyl, indolyl, indolidinyl, isoindolyl, indazolyl, benzotriazolyl, quinolinyl, benzoimidazolyl and benzopyrimidyl groups, the said groups being unsubstituted or substituted with 1 to 3 radicals chosen from a linear or branched C₁-C₄ alkyl radical, a C₁-C₄ monohydroxyalkyl radical, a C₂-C₄ polyhydroxyalkyl radical, a carboxyl radical, an alkoxycarbonyl radical, a halogen atom, an amino radical, an amino radical and a hydroxyl radical.

Group A₄ is understood to mean a C₃-C₇ cycloalkyl radical, a norbornyl radical, carrying or otherwise a double bond and unsubstituted or substituted with 1 or 2 radicals chosen from a linear or branched C₁-C₄ alkyl radical, a C₁-C₄ monohydroxyalkyl radical, a C₂-C₄ polyhydroxyalkyl radical, a carboxyl radical, an alkoxycarbonyl radical, a halogen atom, an amido radical, an amino radical and a hydroxyl radical.

Group A₅ is understood to mean a heterocycle chosen from the dihydrofuranyl, tetrahydrofuranyl, butyrolactonyl, dihydrothiophenyl, tetrahydrothiophenyl, tetrahydrothiophenonyl, iminothiolane, dihydropyrrolyl, pyrrolidinyl, pyrrolidinonyl, imidazolidinonyl, imidazolidinethionyl, oxazolidinyl, oxazolidinonyl, oxazolanethione, thiazolidinyl, isothiazolonyl, mercaptothiazolinyl, pyrazolidinonyl, iminothiolane, dioxolanyl, pentalactone, dioxanyl, dihydropyridinyl, piperidinyl, pentalactam, morpholinyl, pyrazoli(di)nyl, pyrimi(di)nyl, pyrazinyl, piperazinyl and azepinyl rings.

Among these substituents, R₁ preferably represents a hydrogen atom; a methyl, ethyl, isopropyl, allyl, phenyl, benzyl, fluorobenzyl, difluorobenzyl, trifluorobenzyl, chlorobenzyl, bromobenzyl, methoxybenzyl, dimethoxybenzyl, (trifluoromethoxy)benzyl, 3,4-methylenedioxybenzyl, 6-chloropiperonyl, 4-methylthiobenzyl, 4-methylsulphonylbenzyl, 4-acetylaminobenzyl, 4-carboxybenzyl, 1-naphthomethyl or 2-naphthomethyl radical; or a 2-hydroxyethyl, 2-methoxyethyl or 2-ethoxyethyl group.

More preferably still, R₁ represents a hydrogen atom or a methyl radical.

In the compounds of formula (I) according to the invention, R₅ preferably denotes a group A₁, A₂, A₃, A₄ or A₅ as defined above.

Among these substituents, R₅ preferably denotes a methyl, ethyl, propyl, isopropyl, butyl, isobutyl, tert-butyl, pentyl, isopentyl, neopentyl, hexyl; cyclopropyl, cyclobutyl, cyclopentyl, cyclohexyl, vinyl, 1-methylvinyl, 2-methylvinyl, 2,2-dimethylvinyl, allyl, propargyl, chloromethyl, 2-chloroethyl, 2-hydroxyethyl, 2-methoxyethyl, 4-chlorobutyl; phenyl, 4-methylphenyl, 4-ethylphenyl, 4-(trifluoromethyl)phenyl, 4-hydroxyphenyl, 4-methoxyphenyl, 4-ethoxyphenyl, 4-acetoxyphenyl, 4-aminophenyl, 4-fluorophenyl, 4-chlorophenyl, 4-bromophenyl, naphth-1-yl, naphth-2-yl; or benzyl radical.

When R₁ and R₅ form a ring, the said ring is preferably chosen from the 2-oxazolidon-1-yl, 4-methyloxazolidon-1-yl, 5,5-dimethyloxazoline-2,4-dion-1-yl groups.

More preferably still, R₅ represents a methyl, ethyl, propyl, isopropyl, butyl, isobutyl, 2-hydroxyethyl, 2-methoxyethyl, allyl, phenyl, benzyl, cyclopentyl or cyclohexyl radical.

In the compounds of formula (I) according to the invention, R₆ and R₇, which are identical or different, preferably denote a group A₁, A₂, A₃, A₄ or A₅ as defined above.

Among these substituents, R₆ and R₇, which are identical or different, preferably denote a hydrogen atom or a methyl, ethyl, propyl, isopropyl, butyl, isobutyl, tert-butyl, pentyl, isopentyl, neopentyl, hexyl; cyclopentyl, cyclohexyl, allyl, propargyl, 2-chloroethyl, 2-hydroxyethyl, 2-methoxyethyl, 3-chloropropyl, 3-hydroxypropyl, 2,3-dihydroxypropyl, carboxymethyl, phenyl, fluorophenyl, (trifluoromethyl)phenyl, chlorophenyl, bromophenyl, methylphenyl, 4-acetylphenyl, methoxyphenyl, (trifluoromethoxy)phenyl, naphth-1-yl, benzyl, phenethyl or pyrid-3-yl radical.

When R₆ and R₇ form a ring, the said ring is preferably chosen from the pyrrolidin-1-yl, 3-hydroxypyrrolidin-1-yl, 3,4-dihydroxypyrrolidin-1-yl, piperidin-1-yl, 3-hydroxypiperidin-1-yl, 4-hydroxypiperidin-1-yl, 4-morpholin-1-yl, piperazin-1-yl, 4-methylpiperazin-1-yl, 4-(2-hydroxyethyl) piperazin-1-yl groups.

When R₁ and R₆ form a ring, the said ring is preferably chosen from the imidazolidin-2-on-1-yl or tetrahydropyrimidin-2-on-1-yl groups.

More preferably still, R₆ and R₇, which are identical or different, represent a hydrogen atom, or a methyl, ethyl, propyl, isopropyl, butyl, isobutyl, allyl, 2-hydroxyethyl, 2-methoxyethyl, 3-hydroxypropyl, 2,3-dihydroxypropyl, benzyl, cyclopentyl or cyclohexyl radical.

In the compounds of formula (I) according to the invention, R₂ and R₃, which are identical or different, preferably denote a hydrogen or halogen atom; a hydroxyl or amino group; a group A₁, A₄ or A₅ as defined above; a group A₁, A₂, A₃, A₄ or A₅ as defined above and separated from the phenolic nucleus in formula (I) by an oxygen atom or by a group —NH—, -Nalkyl(C₁-C₃)—, —O(CO)—, —NH(CO)—, -Nalkyl(C₁-C₃) (CO)—, -NH[C═NH]—, —NH(CO)NH—, —NH(CO)Nalkyl(C₁-C₃)—, —NH(CO)O—, —NHSO₂—, —NHSO₂NH— or —NHSO₂Nalkyl(C₁-C₃)—.

Among these substituents, R₂ represents more preferably still a hydrogen or chlorine atom; a methyl, hydroxymethyl, methoxymethyl, 2-hydroxyethyl, aminomethyl, methylaminomethyl, hydroxyl, methoxy, acetoxy, amino, methylamino or 2-hydroxyethylamino radical; a group —NH(CO)R₉ in which R₉ represents one of the radicals listed in the group (G1) consisting of a methyl, ethyl, propyl, isopropyl, butyl, isobutyl, tert-butyl, pentyl, isopentyl, neopentyl, hexyl; cyclopropyl, cyclobutyl, cyclopentyl, cyclopentylmethy, 3-cyclopentylpropyl, cyclohexyl, 2-cyclohexylethyl, norborn-2-yl, vinyl, 1-methylvinyl, 2-methylvinyl, 2,2-dimethylvinyl, allyl, 3-butenyl; phenyl, methylphenyl, dimethylphenyl, 2,4,6-trimethylphenyl, 4-ethylphenyl, (trifluoromethyl)phenyl, hydroxyphenyl, methoxyphenyl, ethoxyphenyl, acetoxyphenyl, (trifluoromethoxy)phenyl, aminophenyl, 4-dimethylaminophenyl, fluorophenyl, difluorophenyl, fluoro(trifluoromethyl)phenyl, chlorophenyl, dichlorophenyl, bromophenyl, naphth-1-yl, naphth-2-yl, (2-methoxy)naphth-1-yl, benzyl, 4′-methoxybenzyl, 2′,5′-dimethoxybenzyl, 31,41-dimethoxybenzyl, 4′-fluorobenzyl, 4′-chlorobenzyl, phenethyl, 2-phenylvinyl, (1-naphthyl)methyl, (2-naphthyl)methyl; tetrahydrofuran-2-yl, furan-2-yl, 5-methyl-2-(trifluoromethyl)furan-3-yl, 2-methyl-5-phenylfuran-3-yl, thiophen-2-yl, (thiophen-2-yl)methyl, 3-chlorothiophen-2-yl, 2,5-dichlorothiophen-3-yl, benzothiophen-2-yl, 3-chlorobenzothiophen-2-yl, isoxazol-5-yl, 5-methylisoxazol-3-yl, 3,5-dimethylisoxazol-4-yl, 1,3-dimethylpyrazol-5-yl, 1-ethyl-3-methylpyrazol-5-yl, 1-tert-butyl-3-methylpyrazol-5-yl, 3-tert-butyl-1-methylpyrazol-5-yl, 4-bromo-1-ethyl-3-methylpyrazol-5-yl, indol-3-ylcarboxyl, pyridinyl, chloropyridinyl, dichloropyridinyl, 5-(bromo)pyridin-3-yl, piperazin-2-yl, quinoxal-2-yl; fluoromethyl, difluoromethyl, trifluoromethyl, 1,1,2,2-tetrafluoroethyl, pentafluoroethyl, heptafluoropropyl, 1,1,2,2,3,3,4,4-octafluorobutyl, nonafluorobutyl, chloromethyl, chloroethyl, 1,1-dimethyl-2-chloroethyl, 1,2-dichloroethyl, 1-chloropropyl, 3-chloropropyl, 4-chlorobutyl, hydroxymethyl, methoxymethyl, phenoxymethyl, (4-chlorophenoxy)methyl, benzyloxymethyl, acetoxymethyl, 1,2-dihydroxyethyl, 1-phenoxyethyl, 1-acetoxyethyl, 2-(2-carboxyethoxy)ethyl, 1-phenoxyethyl, 1-acetoxyethyl, methoxycarbonyl, ethoxycarbonyl, (methoxycarbonyl)methyl, 2-carboxyethyl, 2-(methoxycarbonyl)ethyl, 2-carboxycyclopropyl, 2-carboxycyclohexane; methoxy, ethoxy, propoxy, isopropoxy, butoxy, isobutoxy, pentoxy, neopentoxy, hexyloxy, cyclopentyloxy, cyclohexyloxy, vinyloxy, allyloxy, propargyloxy, chloromethoxy, 1-chloroethoxy, 2-methoxyethoxy, 4-chlorobutoxy, phenoxy, 4-methylphenoxy, 4-fluorophenoxy, 4-bromophenoxy, 4-chlorophenoxy, 4-methoxyphenoxy, naphth-2-yloxy, benzyloxy; amino, methylamino, ethylamino, propylamino, isopropylamino, butylamino, cyclohexylamino, allylamino, 2-chloroethylamino, 3-chloropropylamino, carboxymethylamino, phenylamino, fluorophenylamino, (trifluoromethyl)phenylamino, chlorophenylamino, bromophenylamino, 4-acetylphenylamino, methoxyphenylamino, (trifluoromethoxy)phenylamino, naphth-1-ylamino, benzylamino, phenethylamino, pyrid-3-ylamino, dimethylamino, 1-pyrrolidinyl and 4-morpholinyl radical; a group —NHSO₂R₁₀ in which R₁₀ represents one of the radicals listed in the group (G2) consisting of the methyl, trifluoromethyl, ethyl, 2-chloroethyl, propyl, 3-chloropropyl, isopropyl, butyl, thiophen-2-yl, hydroxyl, ethoxy and dimethylamino radicals.

When R₁ and R₂ form a ring, together with the nitrogen atom at the 7-position of the compound of formula (I), the linkage —CH₂CH₂CH₂— is preferred for —R₁R₂—.

When R₂ and R₅ form a ring, together with the nitrogen atom at the 7-position of the compound of formula (I), the linkages —CH₂— or —C(CH₃)₂— are preferred for —R₂R₅—.

When R₂ and R₆ form a ring, together with the nitrogen atom at the 7-position of the compound of formula (I), the linkages —CH₂— or —C(CH₃)₂— are preferred for —R₂R₆—.

More preferably still, R₂ represents a hydrogen atom; a methyl, hydroxymethyl, aminomethyl, hydroxyl, methoxy, amino or methylamino radical; a group methanesulphonylamino; ethanesulphonylamino; dimethylaminosulphonylamino; a group —NH(CO)R₁₁ in which R₁l represents one of the radicals listed in the group (G3) consisting of a methyl, ethyl, propyl, allyl, phenyl, tetrahydrofuran-2-yl, furan-2-yl, thiophen-2-yl, pyridinyl, piperazin-2-yl, fluoromethyl, chloromethyl, 2-chloroethyl, methoxymethyl, acetoxymethyl, 1,2-dihydroxyethyl, methoxycarbonyl, 2-carboxyethyl, methoxy, ethoxy, propoxy, allyloxy, 2-chloroethoxy, 2-methoxyethoxy, amino, ethylamino, allylamino, 2-chloroethylamino, pyridylamino, dimethylamino, 1-pyrrolidinyl and 4-morpholinyl radical.

Among these substituents, R₃ preferably represents a hydrogen or chlorine atom; a methyl, ethyl, hydroxymethyl, 2-hydroxyethyl, methoxymethyl, aminomethyl, methylaminomethyl, hydroxyl, methoxy, acetoxy, amino, methylamino, 2-hydroxyethylamino, N-piperidino or N-morpholino radical; a group —NH(CO)R₁₂ in which R₁₂ represents one of the radicals listed in the group (G1) defined above; or a group —NHSO₂R₁₃ in which R₁₃ represents one of the radicals listed in the group (G2) defined above.

More preferably still, R₃ represents a hydrogen or chlorine atom; a methyl, hydroxymethyl, aminomethyl, hydroxyl, methoxy, amino or methylamino radical; a group methanesulphonylamino; ethanesulphonylamino; dimethylaminosulphonylamino; a group —NH(CO)R₁₄ in which R₁₄ represents one of the radicals listed in the group (G3) defined above.

According to the invention, R₄ is preferably chosen from a hydrogen or halogen atom; a group A₁, A₄ or A₅ as defined above; a group A₁, A₂, A₃, A₄ or A₅ as defined above and separated from the phenolic nucleus of the compounds of formula (I) by an oxygen or sulphur atom or by a group —NH—, -Nalkyl(C₁-C₃)—, —O(CO)—, —NH(CO)—, -Nalkyl(C₁-C₃) (CO)—, —NH[C═NH]—, —NH(CO)NH—, —NH(CO)Nalkyl(C₁-C₃)—or —NH(CO)O—.

Among these substituents, R₄ preferably represents a hydrogen, chlorine, fluorine or bromine atom; a methyl, trifluoromethyl, allyl, hydroxymethyl, methoxymethyl, 2-hydroxyethyl, aminomethyl, methylaminomethyl, methoxy, acetoxy or methylamino radical; or a group —NH(CO)R₁₅ in which R₁₅ represents one of the radicals listed in the group (G1) defined above.

More preferably still, R₄ represents a hydrogen, chlorine or fluorine atom; a methyl, hydroxymethyl, aminomethyl, methoxy or methylamino radical; a group —NH(CO)R₁₆ in which R₁₆ represents one of the radicals listed in the group (G3) defined above.

In compounds of the formula (I) according to the invention, Y is preferably chosen from a hydrogen, chlorine, fluorine or bromine atom; a group methoxy, ethoxy, propoxy, benzyloxy, phenoxy, —OCH₂CH₂OCH₃; —OCH₂CH₂OCH₃; —OCH₂CH₂N(CH₃)₂; —OCH₂(CO)OH, —OCH₂(CO)OCH₃, —OCH₂(CO)OC₂H₅, —SCH₂CH₂CO₂H, or —NHSO₂CH₃; it being understood that Y cannot represent a group —NHSO₂CH₃ when R₂ represents a hydroxyl radical.

More preferably still, Y represents a hydrogen or chlorine atom or a group methoxy, —OCH₂(CO)OH, or —OCH₂(CO)OCH₃.

Among the compounds of formula (I) in accordance with the invention, there are particularly preferred those in which:

i)

R₁ represents a hydrogen atom;

X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;

R₂ represents a hydroxyl, amino, methylamino, 2-hydroxyethylamino or diethylamino radical; a group —NH(CO)R₁₇ in which R₁₇ represents one of the radicals listed in the group (G4) consisting of a methyl, methoxymethyl, 2-carboxyethyl, methoxy, amino, ethylamino and 1-pyrrolidinyl radical; a methanesulphonylamino, ethanesulphonylamino or dimethylaminosulphonylamino group;

R₃ represents a hydrogen or chlorine atom, or a methyl group;

R₄ represents a hydrogen, chlorine or fluorine atom or a methyl group;

Y represents a hydrogen or chlorine atom; a methoxy or —OCH₂(CO)OCH₃ group;

ii)

R₁ represents a hydrogen atom;

X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;

R₂ represents a hydrogen atom; or a methyl radical;

R₃ represents a hydroxyl, amino, methylamino, 2-hydroxyethylamino, diethylamino, methanesulphonylamino, ethanesulphonylamino or dimethylaminosulphonylamino radical; a group —NH(CO)R₁₈ in which R₁₈ represents one of the radicals listed in the group (G4) defined above;

R₄ represents a hydrogen, chlorine or fluorine atom; or a methyl group;

Y represents a hydrogen or chlorine atom; a methoxy or —OCH₂(CO)OCH₃ group;

iii)

R₁ represents a hydrogen atom;

X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;

R₂ represents a hydrogen atom; or a methyl radical;

R₃ represents a hydrogen or chlorine atom; a methyl radical; or a methoxy or methylamino group;

R₄ represents a group —NH(CO)R₁₉ in which R₁₉ represents one of the radicals listed in the group (G4) defined above;

Y represents a hydrogen or chlorine atom; or a methoxy or —OCH₂(CO)OCH₃ group;

iv)

R₁ represents a hydrogen atom;

X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;

R₂ represents a hydrogen atom; or a methyl radical;

R₃ represents a hydrogen atom; or a methyl or ethyl group;

R₄ represents a hydrogen, chlorine or fluorine atom;

Y represents a hydrogen or chlorine atom; or a group —OCH₂(CO)OCH₃.

Among the compounds of formula (I) in accordance with the invention, there may be mentioned:

(2-Hydroxyphenyl)carbamic acid methyl ester,

(2-Hydroxyphenyl)carbamic acid ethyl ester,

(2-Hydroxyphenyl)carbamic acid 2-hydroxyethyl ester,

(2-Hydroxyphenyl)carbamic acid isopropyl ester,

1-(2-Hydroxyphenyl)urea,

3-(2-Hydroxyphenyl)-1-methylurea,

3-(2-Hydroxyphenyl)-1-ethylurea,

3-(2-Hydroxyphenyl)-1-(2-hydroxyethyl)urea,

3-(2-Hydroxyphenyl)-1-isopropylurea,

3-(2-Hydroxyphenyl)-1,1-dimethylurea,

3-(2-Hydroxyphenyl)-1,1-diethylurea,

3-(2-Hydroxyphenyl)-1,1-di(2-hydroxyethyl)urea,

(2-Hydroxy-5-chlorophenyl)carbamic acid methyl ester,

(2-Hydroxy-5-chlorophenyl)carbamic acid ethyl ester,

(2-Hydroxy-5-chlorophenyl)carbamic acid 2-hydroxyethyl ester,

(2-Hydroxy-5-chlorophenyl)carbamic acid isopropyl ester,

1-(2-Hydroxy-5-chlorophenyl)urea,

3-(2-Hydroxy-5-chlorophenyl)-1-methylurea,

3-(2-Hydroxy-5-chlorophenyl)-1-ethylurea,

3-(2-Hydroxy-5-chlorophenyl)-1-(2-hydroxyethyl)urea,

3-(2-Hydroxy-5-chlorophenyl)-1-isopropylurea,

3-(2-Hydroxy-5-chlorophenyl)-1,1-dimethylurea,

3-(2-Hydroxy-5-chlorophenyl)-1,1-diethylurea,

3-(2-Hydroxy-5-chlorophenyl)-1,1-di(2-hydroxyethyl)urea,

(2-Hydroxy-5-methoxyphenyl)carbamic acid methyl ester,

(2-Hydroxy-5-methoxyphenyl)carbamic acid ethyl ester,

(2-Hydroxy-5-methoxyphenyl)carbamic acid 2-hydroxyethyl ester,

(2-Hydroxy-5-methoxyphenyl)carbamic acid isopropyl ester,

3-(2-Hydroxy-5-methoxyphenyl)-1-(2-hydroxyethyl)urea,

3-(2-Hydroxy-5-methoxyphenyl)-1,1-di(2-hydroxyethyl)urea,

(2-Hydroxy-6-aminophenyl)carbamic acid methyl ester,

(2-Hydroxy-6-aminophenyl)carbamic acid ethyl ester,

(2-Hydroxy-6-aminophenyl)carbamic acid 2-hydroxyethyl ester,

(2-Hydroxy-6-aminophenyl)carbamic acid isopropyl ester,

1-(2-Hydroxy-6-aminophenyl)urea,

3-(2-Hydroxy-6-aminophenyl)-1-methylurea,

3-(2-Hydroxy-6-aminophenyl)-1-ethylurea,

3-(2-Hydroxy-6-aminophenyl)-1-(2-hydroxyethyl)urea,

3-(2-Hydroxy-6-aminophenyl)-1-isopropylurea,

3-(2-Hydroxy-6-aminophenyl)-1,1-dimethylurea,

3-(2-Hydroxy-6-aminophenyl)-1,1-diethylurea,

3-(2-Hydroxy-6-aminophenyl)-1,1-di(2-hydroxyethyl)urea,

(2-Hydroxy-4-aminophenyl)carbamic acid methyl ester,

(2-Hydroxy-4-aminophenyl)carbamic acid ethyl ester,

(2-Hydroxy-4-aminophenyl)carbamic acid 2-hydroxyethyl ester,

(2-Hydroxy-4-aminophenyl)carbamic acid isopropyl ester,

1-(2-Hydroxy-4-aminophenyl)urea,

3-(2-Hydroxy-4-aminophenyl)-1-methylurea,

3-(2-Hydroxy-4-aminophenyl)-1-ethylurea,

3-(2-Hydroxy-4-aminophenyl)-1-(2-hydroxyethyl)urea,

3-(2-Hydroxy-4-aminophenyl)-1-isopropylurea,

3-(2-Hydroxy-4-aminophenyl)-1,1-dimethylurea,

3-(2-Hydroxy-4-aminophenyl)-1,1-diethylurea,

3-(2-Hydroxy-4-aminophenyl)-1,1-di(2-hydroxyethyl)urea,

Pyrrolidine-1-carboxylic acid (4-amino-2-hydroxyphenyl)amide,

3-Hydroxypyrrolidine-1-carboxylic acid (4-amino-2-hydroxyphenyl)amide,

(2-Hydroxy-4-methylaminophenyl)carbamic acid methyl ester,

(2-Hydroxy-4-methylaminophenyl)carbamic acid ethyl ester,

(2-Hydroxy-4-methylaminophenyl)carbamic acid 2-hydroxyethyl ester,

(2-Hydroxy-4-methylaminophenyl)carbamic acid isopropyl ester,

1-(2-Hydroxy-4-methylaminophenyl)urea,

3-(2-Hydroxy-4-methylaminophenyl)-1-methylurea,

3-(2-Hydroxy-4-methylaminophenyl)-1-ethylurea,

3-(2-Hydroxy-4-methylaminophenyl)-1-(2-hydroxyethyl)urea,

3-(2-Hydroxy-4-methylaminophenyl)-1-isopropylurea,

3-(2-Hydroxy-4-methylaminophenyl)-1,1-dimethylurea,

3-(2-Hydroxy-4-methylaminophenyl)-1,1-diethylurea,

3-(2-Hydroxy-4-methylaminophenyl)-1,1-di(2-hydroxyethyl)urea,

(2-Hydroxy-4-(2-hydroxyethyl)aminophenyl)carbamic acid methyl ester,

(2-Hydroxy-4-(2-hydroxyethyl)aminophenyl)carbamic acid ethyl ester,

(2-Hydroxy-4-(2-hydroxyethyl)aminophenyl)carbamic acid 2-hydroxyethyl ester,

(2-Hydroxy-4-(2-hydroxyethyl)aminophenyl)carbamic acid isopropyl ester,

1-(2-Hydroxy-4-(2-hydroxyethyl)aminophenyl)urea,

3-(2-Hydroxy-4-(2-hydroxyethyl)aminophenyl)-1-methylurea,

3-(2-Hydroxy-4-(2-hydroxyethyl)aminophenyl)-1-ethylurea,

3-(2-Hydroxy-4-(2-hydroxyethyl)aminophenyl)-1-(2-hydroxyethyl)urea,

3-(2-Hydroxy-4-(2-hydroxyethyl)aminophenyl)-1-isopropylurea,

3-(2-Hydroxy-4-(2-hydroxyethyl)aminophenyl)-1,1-dimethylurea,

3-(2-Hydroxy-4-(2-hydroxyethyl)aminophenyl)-1,1-diethylurea,

3-(2-Hydroxy-4-(2-hydroxyethyl)aminophenyl)-1,1-di(2-hydroxyethyl)urea,

(2-Hydroxy-4-amino-5-chlorophenyl)carbamic acid methyl ester,

(2-Hydroxy-4-amino-5-chlorophenyl)carbamic acid ethyl ester,

(2-Hydroxy-4-amino-5-chlorophenyl)carbamic acid 2-hydroxyethyl ester,

(2-Hydroxy-4-amino-5-chlorophenyl)carbamic acid isopropyl ester,

1-(2-Hydroxy-4-amino-5-chlorophenyl)urea,

3-(2-Hydroxy-4-amino-5-chlorophenyl)-1-methylurea,

3-(2-Hydroxy-4-amino-5-chlorophenyl)-1-ethylurea,

3-(2-Hydroxy-4-amino-5-chlorophenyl)-1-(2-hydroxyethyl)urea,

3-(2-Hydroxy-4-amino-5-chlorophenyl)-1-isopropylurea,

3-(2-Hydroxy-4-amino-5-chlorophenyl)-1,1-dimethylurea,

3-(2-Hydroxy-4-amino-5-chlorophenyl)-1,1-diethylurea,

3-(2-Hydroxy-4-amino-5-chlorophenyl)-1,1-di(2-hydroxyethyl)urea,

(2-Hydroxy-4-methylamino-5-chlorophenyl)carbamic acid methyl ester,

(2-Hydroxy-4-methylamino-5-chlorophenyl)carbamic acid ethyl ester,

(2-Hydroxy-4-methylamino-5-chlorophenyl)carbamic acid 2-hydroxyethyl ester,

(2-Hydroxy-4-methylamino-5-chlorophenyl)carbamic acid isopropyl ester,

1-(2-Hydroxy-4-methylamino-5-chlorophenyl)urea,

3-(2-Hydroxy-4-methylamino-5-chlorophenyl)-1-methylurea,

3-(2-Hydroxy-4-methylamino-5-chlorophenyl)-1-ethylurea,

3-(2-Hydroxy-4-methylamino-5-chlorophenyl)-1-(2-hydroxyethyl)urea,

3-(2-Hydroxy-4-methylamino-5-chlorophenyl)-1-isopropylurea,

3-(2-Hydroxy-4-methylamino-5-chlorophenyl)-1,1-dimethylurea,

3-(2-Hydroxy-4-methylamino-5-chlorophenyl)-1,1-diethylurea,

3-(2-Hydroxy-4-methylamino-5-chlorophenyl)-1,1-di(2-hydroxyethyl)urea,

(2-Hydroxy-4-(2-hydroxyethyl)amino-5-chlorophenyl)carbamic acid methyl ester,

(2-Hydroxy-4-(2-hydroxyethyl)amino-5-chlorophenyl)carbamic acid ethyl ester,

(2-Hydroxy-4-(2-hydroxyethyl)amino-5-chlorophenyl)carbamic acid 2-hydroxyethyl ester,

(2-Hydroxy-4-(2-hydroxyethyl)amino-5-chlorophenyl)carbamic acid isopropyl ester,

1-(2-Hydroxy-4-(2-hydroxyethyl)amino-5-chlorophenyl)urea,

3-(2-Hydroxy-4-(2-hydroxyethyl)amino-5-chlorophenyl)-1-methylurea,

3-(2-Hydroxy-4-(2-hydroxyethyl)amino-5-chlorophenyl)-1-ethylurea,

3-(2-Hydroxy-4-(2-hydroxyethyl)amino-5-chlorophenyl)-1-(2-hydroxyethyl)urea,

3-(2-Hydroxy-4-(2-hydroxyethyl)amino-5-chlorophenyl)-1-isopropylurea,

3-(2-Hydroxy-4-(2-hydroxyethyl)amino-5-chlorophenyl)-1,1-dimethylurea,

3-(2-Hydroxy-4-(2-hydroxyethyl)amino-5-chlorophenyl)-1,1-diethylurea,

3-(2-Hydroxy-4-(2-hydroxyethyl)amino-5-chlorophenyl)-1,1-di(2-hydroxyethyl)urea,

(2-Hydroxy-4-amino-5-methoxyphenyl)carbamic acid methyl ester,

(2-Hydroxy-4-amino-5-methoxyphenyl)carbamic acid ethyl ester,

(2-Hydroxy-4-amino-5-methoxyphenyl)carbamic acid 2-hydroxyethyl ester,

(2-Hydroxy-4-amino-5-methoxyphenyl)carbamic acid isopropyl ester,

3-(2-Hydroxy-4-amino-5-methoxyphenyl)1-(2-hydroxyethyl)urea,

3-(2-Hydroxy-4-amino-5-methoxyphenyl)-1,1-di(2-hydroxyethyl)urea,

(2-Hydroxy-4-methylamino-5-methoxyphenyl)carbamic acid methyl ester,

(2-Hydroxy-4-methylamino-5-methoxyphenyl)carbamic acid ethyl ester,

(2-Hydroxy-4-methylamino-5-methoxyphenyl)carbamic acid 2-hydroxyethyl ester,

(2-Hydroxy-4-methylamino-5-methoxyphenyl)carbamic acid isopropyl ester,

1-(2-Hydroxy-4-methylamino-5-methoxyphenyl)urea,

3-(2-Hydroxy-4-methylamino-5-methoxyphenyl)-1-methylurea,

3-(2-Hydroxy-4-methylamino-5-methoxyphenyl)-1-ethylurea,

3-(2-Hydroxy-4-methylamino-5-methoxyphenyl)-1-(2-hydroxyethyl)urea,

3-(2-Hydroxy-4-methylamino-5-methoxyphenyl)-1-isopropylurea,

3-(2-Hydroxy-4-methylamino-5-methoxyphenyl)-1,1-dimethylurea,

3-(2-Hydroxy-4-methylamino-5-methoxyphenyl)-1,1-diethylurea,

3-(2-Hydroxy-4-methylamino-5-methoxyphenyl)-1,1-di(2-hydroxyethyl)urea,

(2-Hydroxy-4-(2-hydroxyethyl)amino-5-methoxyphenyl)carbamic acid methyl ester,

(2-Hydroxy-4-(2-hydroxyethyl)amino-5-methoxyphenyl)carbamic acid ethyl ester,

(2-Hydroxy-4-(2-hydroxyethyl)amino-5-methoxyphenyl)carbamic acid 2-hydroxyethyl ester,

(2-Hydroxy-4-(2-hydroxyethyl)amino-5-methoxyphenyl)carbamic acid isopropyl ester,

1-(2-Hydroxy-4-(2-hydroxyethyl)amino-5-methoxyphenyl)urea,

3-(2-Hydroxy-4-(2-hydroxyethyl)amino-5-methoxyphenyl)-1-methylurea,

3-(2-Hydroxy-4-(2-hydroxyethyl)amino-5-methoxyphenyl)-1-ethylurea,

3-(2-Hydroxy-4-(2-hydroxyethyl)amino-5-methoxyphenyl)-1-(2-hydroxyethyl)urea,

3-(2-Hydroxy-4-(2-hydroxyethyl)amino-5-methoxyphenyl)-1-isopropylurea,

3-(2-Hydroxy-4-(2-hydroxyethyl)amino-5-methoxyphenyl)-1,1-dimethylurea,

3-(2-Hydroxy-4-(2-hydroxyethyl)amino-5-methoxyphenyl)-1,1-diethylurea,

3-(2-Hydroxy-4-(2-hydroxyethyl)amino-5-methoxyphenyl)-1,1-di(2-hydroxyethyl)urea,

(2-Hydroxy-3-methoxycarbonylaminophenyl)carbamic acid methyl ester,

(2-Hydroxy-3-ethoxycarbonylaminophenyl)carbamic acid ethyl ester,

(2-Hydroxy-3-(2-hydroxyethoxy)carbonylaminophenyl)-carbamic acid 2-hydroxyethyl ester,

(2-Hydroxy-3-isopropoxycarbonylaminophenyl)carbamic acid isopropyl ester,

1-(2-Hydroxy-3-ureidophenyl)urea,

3-(2-Hydroxy-3-(3-methylureido)phenyl)-1-methylurea,

3-(2-Hydroxy-3-(3-ethylureido)phenyl)-1-ethylurea,

3-(2-Hydroxy-3-(3-(2-hydroxyethyl)ureido)phenyl)-1-(2-hydroxyethyl)urea,

3-(2-Hydroxy-3-(3-isopropylureido)phenyl)-1-isopropylurea,

3-(2-Hydroxy-3-(3,3-dimethylureido)phenyl)-1,1-dimethylurea,

and their addition salts with an acid.

Preferably, the compounds of formula (I) are chosen from:

(2-Hydroxy-4-aminophenyl)carbamic acid methyl ester,

(2-Hydroxy-4-aminophenyl)carbamic acid ethyl ester,

(2-Hydroxy-4-aminophenyl)carbamic acid 2-hydroxyethyl ester,

(2-Hydroxy-4-aminophenyl)carbamic acid isopropyl ester,

1-(2-Hydroxy-4-aminophenyl)urea,

3-(2-Hydroxy-4-aminophenyl)-1-methylurea,

3-(2-Hydroxy-4-aminophenyl)-1-ethylurea,

3-(2-Hydroxy-4-aminophenyl)-1-(2-hydroxyethyl)urea,

3-(2-Hydroxy-4-aminophenyl)-1-isopropylurea,

3-(2-Hydroxy-4-aminophenyl)-1,1-dimethylurea,

(2-Hydroxy-4-methylaminophenyl)carbamic acid methyl ester,

(2-Hydroxy-4-methylaminophenyl)carbamic acid ethyl ester,

(2-Hydroxy-4-methylaminophenyl)carbamic acid 2-hydroxyethyl ester,

(2-Hydroxy-4-methylaminophenyl)carbamic acid isopropyl ester,

1-(2-Hydroxy-4-methylaminophenyl)urea,

3-(2-Hydroxy-4-methylaminophenyl)-1-methylurea,

3-(2-Hydroxy-4-methylaminophenyl)-1-ethylurea,

3-(2-Hydroxy-4-methylaminophenyl)-1-(2-hydroxyethyl)urea,

3-(2-Hydroxy-4-methylaminophenyl)-1-isopropylurea,

3-(2-Hydroxy-4-methylaminophenyl)-1,1-dimethylurea,

(2-Hydroxy-4-(2-hydroxyethyl)aminophenyl)carbamic acid methyl ester,

(2-Hydroxy-4-(2-hydroxyethyl)aminophenyl)carbamic acid ethyl ester,

(2-Hydroxy-4-(2-hydroxyethyl)aminophenyl)carbamic acid 2-hydroxyethyl ester,

(2-Hydroxy-4-(2-hydroxyethyl)aminophenyl)carbamic acid isopropyl ester,

1-(2-Hydroxy-4-(2-hydroxyethyl)aminophenyl)urea,

3-(2-Hydroxy-4-(2-hydroxyethyl)aminophenyl)-1-methylurea,

3-(2-Hydroxy-4-(2-hydroxyethyl)aminophenyl)-1-ethylurea,

3-(2-Hydroxy-4-(2-hydroxyethyl)aminophenyl)-1-(2-hydroxyethyl)urea,

3-(2-Hydroxy-4-(2-hydroxyethyl)aminophenyl)-1-isopropylurea,

3-(2-Hydroxy-4-(2-hydroxyethyl)aminophenyl)-1,1-dimethylurea,

(2-Hydroxy-4-amino-5-chlorophenyl)carbamic acid methyl ester,

(2-Hydroxy-4-amino-5-chlorophenyl)carbamic acid ethyl ester,

(2-Hydroxy-4-amino-5-chlorophenyl)carbamic acid 2-hydroxyethyl ester,

(2-Hydroxy-4-amino-5-chlorophenyl)carbamic acid isopropyl ester,

1-(2-Hydroxy-4-amino-5-chlorophenyl)urea,

3-(2-Hydroxy-4-amino-5-chlorophenyl)-1-methylurea,

3-(2-Hydroxy-4-amino-5-chlorophenyl)-1-ethylurea,

3-(2-Hydroxy-4-amino-5-chlorophenyl)1-(2-hydroxyethyl)urea,

3-(2-Hydroxy-4-amino-5-chlorophenyl)-1-isopropylurea,

3-(2-Hydroxy-4-amino-5-chlorophenyl)-1,1-dimethylurea,

(2-Hydroxy-4-methylamino-5-chlorophenyl)carbamic acid methyl ester,

(2-Hydroxy-4-methylamino-5-chlorophenyl)carbamic acid ethyl ester,

(2-Hydroxy-4-methylamino-5-chlorophenyl)carbamic acid 2-hydroxyethyl ester,

(2-Hydroxy-4-methylamino-5-chlorophenyl)carbamic acid isopropyl ester,

1-(2-Hydroxy-4-methylamino-5-chlorophenyl)urea,

3-(2-Hydroxy-4-methylamino-5-chlorophenyl)-1-methylurea,

3-(2-Hydroxy-4-methylamino-5-chlorophenyl)-1-ethylurea,

3-(2-Hydroxy-4-methylamino-5-chlorophenyl)-1-(2-hydroxyethyl)urea,

3-(2-Hydroxy-4-methylamino-5-chlorophenyl)-1-isopropylurea,

3-(2-Hydroxy-4-methylamino-5-chlorophenyl)-1,1-dimethylurea,

(2-Hydroxy-4-(2-hydroxyethyl)amino-5-chlorophenyl)carbamic acid methyl ester,

(2-Hydroxy-4-(2-hydroxyethyl)amino-5-chlorophenyl)carbamic acid ethyl ester,

(2-Hydroxy-4-(2-hydroxyethyl)amino-5-chlorophenyl)carbamic acid 2-hydroxyethyl ester,

(2-Hydroxy-4-(2-hydroxyethyl)amino-5-chlorophenyl)carbamic acid isopropyl ester,

1-(2-Hydroxy-4-(2-hydroxyethyl)amino-5-chlorophenyl)urea,

3-(2-Hydroxy-4-(2-hydroxyethyl)amino-5-chlorophenyl)-1-methylurea,

3-(2-Hydroxy-4-(2-hydroxyethyl)amino-5-chlorophenyl)-1-ethylurea,

3-(2-Hydroxy-4-(2-hydroxyethyl)amino-5-chlorophenyl)-1-(2-hydroxyethyl)urea,

3-(2-Hydroxy-4-(2-hydroxyethyl)amino-5-chlorophenyl)-1-isopropylurea,

3-(2-Hydroxy-4-(2-hydroxyethyl)amino-5-chlorophenyl)-1,1-dimethylurea,

(2-Hydroxy-4-amino-5-methoxyphenyl)carbamic acid methyl ester,

(2-Hydroxy-4-amino-5-methoxyphenyl)carbamic acid ethyl ester,

(2-Hydroxy-4-amino-5-methoxyphenyl)carbamic acid 2-hydroxyethyl ester,

(2-Hydroxy-4-amino-5-methoxyphenyl)carbamic acid isopropyl ester,

3-(2-Hydroxy-4-amino-5-methoxyphenyl)-1-(2-hydroxyethyl)urea,

(2-Hydroxy-4-methylamino-5-methoxyphenyl)carbamic acid methyl ester,

(2-Hydroxy-4-methylamino-5-methoxyphenyl)carbamic acid ethyl ester,

(2-Hydroxy-4-methylamino-5-methoxyphenyl)carbamic acid 2-hydroxyethyl ester,

(2-Hydroxy-4-methylamino-5-methoxyphenyl)carbamic acid isopropyl ester,

1-(2-Hydroxy-4-methylamino-5-methoxyphenyl)urea,

3-(2-Hydroxy-4-methylamino-5-methoxyphenyl)-1-methylurea,

3-(2-Hydroxy-4-methylamino-5-methoxyphenyl)-1-ethylurea,

3-(2-Hydroxy-4-methylamino-5-methoxyphenyl)-1-(2-hydroxyethyl)urea,

3-(2-Hydroxy-4-methylamino-5-methoxyphenyl)-1-isopropylurea,

3-(2-Hydroxy-4-methylamino-5-methoxyphenyl)-1,1-dimethylurea,

(2-Hydroxy-4-(2-hydroxyethyl)amino-5-methoxyphenyl)carbamic acid methyl ester,

(2-Hydroxy-4-(2-hydroxyethyl)amino-5-methoxyphenyl)carbamic acid ethyl ester,

(2-Hydroxy-4-(2-hydroxyethyl)amino-5-methoxyphenyl)carbamic acid 2-hydroxyethyl ester,

(2-Hydroxy-4-(2-hydroxyethyl)amino-5-methoxyphenyl)carbamic acid isopropyl ester,

1-(2-Hydroxy-4-(2-hydroxyethyl)amino-5-methoxyphenyl)urea,

3-(2-Hydroxy-4-(2-hydroxyethyl)amino-5-methoxyphenyl)-1-methylurea,

3-(2-Hydroxy-4-(2-hydroxyethyl)amino-5-methoxyphenyl)-1-ethylurea,

3-(2-Hydroxy-4-(2-hydroxyethyl)amino-5-methoxyphenyl)-1-(2-hydroxyethyl)urea,

3-(2-Hydroxy-4-(2-hydroxyethyl)amino-5-methoxyphenyl)-1-isopropylurea,

3-(2-Hydroxy-4-(2-hydroxyethyl)amino-5-methoxyphenyl)-1,1-dimethylurea,

and their addition salts with an acid.

More preferably still, the compounds of formula (I) are chosen from:

(2-Hydroxy-4-aminophenyl)carbamic acid methyl ester,

(2-Hydroxy-4-aminophenyl)carbamic acid ethyl ester,

(2-Hydroxy-4-aminophenyl)carbamic acid 2-hydroxyethyl ester,

(2-Hydroxy-4-aminophenyl)carbamic acid isopropyl ester,

1-(2-Hydroxy-4-aminophenyl)urea,

3-(2-Hydroxy-4-aminophenyl)-1-methylurea,

3-(2-Hydroxy-4-aminophenyl)-1-ethylurea,

3-(2-Hydroxy-4-aminophenyl)-1-(2-hydroxyethyl)urea,

3-(2-Hydroxy-4-aminophenyl)-1-isopropylurea,

3-(2-Hydroxy-4-aminophenyl)-1,1-dimethylurea,

(2-Hydroxy-4-amino-5-chlorophenyl)carbamic acid methyl ester,

(2-Hydroxy-4-amino-5-chlorophenyl)carbamic acid ethyl ester,

(2-Hydroxy-4-amino-5-chlorophenyl)carbamic acid 2-hydroxyethyl ester,

(2-Hydroxy-4-amino-5-chlorophenyl)carbamic acid isopropyl ester,

1-(2-Hydroxy-4-amino-5-chlorophenyl)urea,

3-(2-Hydroxy-4-amino-5-chlorophenyl)-1-methylurea,

3-(2-Hydroxy-4-amino-5-chlorophenyl)-1-ethylurea,

3-(2-Hydroxy-4-amino-5-chlorophenyl)-1-(2-hydroxyethyl)urea,

3-(2-Hydroxy-4-amino-5-chlorophenyl)-1-isopropylurea,

3-(2-Hydroxy-4-amino-5-chlorophenyl)-1,1-dimethylurea,

(2-Hydroxy-4-amino-5-methoxyphenyl)carbamic acid methyl ester,

(2-Hydroxy-4-amino-5-methoxyphenyl)carbamic acid ethyl ester,

(2-Hydroxy-4-amino-5-methoxyphenyl)carbamic acid 2-hydroxyethyl ester,

(2-Hydroxy-4-amino-5-methoxyphenyl)carbamic acid isopropyl ester,

3-(2-Hydroxy-4-amino-5-methoxyphenyl)1-(2-hydroxyethyl)urea,

and their addition salts with an acid.

The compounds of formula (I) in accordance with the invention are known compounds which may be prepared according to conventional methods well known in the state of the art and which are described for example in patent applications and patents JP 43008269, DE 2 156 480, U.S. Pat. No. 4,310,527, JP 60108844, CS 235432, U.S. Pat. No. 3,767,412, JP 02220047, JP 61035444, JP 60237444, DE 3 524 519, EP 0 086 126, U.S. Pat. No. 2,795,610 or in J. Heterocyclic Chem. 1983,20, 1423, J. Mol. Struct. 1995, 344, 251.

The compound(s) of formula (I) in accordance with the invention and/or its (their) addition salt(s) with an acid preferably represent(s) from 0.0005 to 12% by weight approximately of the total weight of the dyeing composition and more preferably still from 0.005 to 6% by weight approximately of this weight.

According to a particularly preferred embodiment of the invention, the preferred dyeing compositions contain at least the ethyl ester of (2-hydroxy-4-aminophenyl)carbamic acid or one of its addition salts with an acid as compound of formula (I) in combination with at least one pyrazolopyrimidine chosen from 5-methylpyrazolo[1,5-a]pyrimidine-3,7-diamine; pyrazolo[1,5-a]pyrimidine-3,7-diamine; 2-methylpyrazolo[1,5-a]pyrimidine-3,7-diamine; 3-amino-5-methyl-7-imidazolylpropylaminopyrazolo[1,5-a]-pyrimidine; 3-amino-5,6-dimethyl-7-imidazolylpropylaminopyrazolo[1,5-a]pyrimidine; 2-methoxy-5,7-dimethylpyrazolo[1,5-a]pyrimidin-3-ylamine; 3-amino-7-imidazolylpropylaminopyrazolo[1,5-a]-pyrimidine; and their addition salts with an acid.

Among these combinations, the combination of 5-methylpyrazolo[1,5-a]pyrimidine-3,7-diamine or of one of its addition salts with an acid and the ethyl ester of (2-hydroxy-4-aminophenyl)carbamic acid or one of its addition salts with an acid is particularly preferred.

In addition to the pyrazolopyrimidines used as oxidation bases, the oxidation dyeing composition in accordance with the invention may contain one or more additional oxidation bases which are preferably chosen from the oxidation bases conventionally used in oxidation dyeing and among which there may be mentioned para-phenylenediamines, bisphenylalkylenediamines, para-aminophenols, ortho-aminophenols and heterocyclic bases other than pyrazolopyrimidines.

Among the para-phenylenediamines, there may be mentioned more particularly by way of example para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(D-hydroxypropyl)-para-phenylenediamine, 2-hydroxymethyl-para-phenylene-diamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

Among the para-phenylenediamines mentioned above, there are most particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylene-diamine, 2,6-dimethyl-para-phenylenediamine, 2,6-diethyl-para-phenylenediamine, 2,3-dimethyl-para-phenylenediamine, N,N-bis (β-hydroxyethyl)-para-phenylenediamine, 2-chloro-para-phenylenediamine, 2-β-acetylaminoethyloxy-para-phenylenediamine, and their addition salts with an acid.

Among the bisphenylalkylenediamines, there may be mentioned more particularly by way of example N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their addition salts with an acid.

Among the para-aminophenols, there may be mentioned more particularly by way of example para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols, there may be mentioned more particularly by way of example 2-aminophenol, 2-amino-5-methylphenol, 2-amino-6-methylphenol, 5-acetamido-2-aminophenol, and their addition salts with an acid.

Among the heterocyclic bases, there may be mentioned more particularly by way of example pyridine derivatives, pyrimidine derivatives and pyrazole derivatives.

Among the pyridine derivatives, there may be mentioned more particularly the compounds described for example in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Among the pyrimidine derivatives, there may be mentioned more particularly the compounds described for example in German Patent DE 2,359,399 or Japanese Patents JP 88-169,571 and JP 91-10659 or Patent Application WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine.

Among the pyrazole derivatives, there may be mentioned more particularly the compounds described in Patents DE 3,843,892, DE 4,133,957 and Patent Applications WO 94/08969, WO 94/08970, FR-A-2,733,749 and DE 195 43 9.88 such as 4,5-diamino-1-methylpyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(P-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(P-hydroxyethyl)amino-1-methylpyrazole, and their addition salts with an acid.

The additional oxidation base(s) preferably represent(s) from 0.0005 to 12% by weight approximately of the total weight of the dyeing composition, and more preferably still from 0.005 to 6% by weight approximately of this weight.

The dyeing composition in accordance with the invention may also contain, in addition to the compound(s) of formula (I) above, one or more additional couplers which may be chosen from the couplers conventionally used in oxidation dyeing and among which there may be mentioned in particular meta-phenylenediamines, meta-aminophenols, meta-diphenols and heterocyclic couplers such as, for example, indole derivatives, indoline derivatives, pyridine derivatives and pyrazolones, and their addition salts with an acid.

These couplers are more particularly chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, and their addition salts with an acid.

When they are present, these additional couplers preferably represent from 0.0001 to 10% by weight approximately of the total weight of the dyeing composition and more preferably still from 0.005 to 5% by weight approximately of this weight.

In general, the addition salts with an acid which can be used in the context of the dyeing compositions of the invention (pyrazolopyrimidines, compounds of formula (I), oxidation bases and additional couplers) are chosen in particular from hydrochlorides, hydrobromides, sulphates, citrates, succinates, tartrates, lactates and acetates.

The medium appropriate for dyeing (or carrier) generally consists of water or of a mixture of water and at least one organic solvent for solubilizing the compounds which might not be sufficiently soluble in water. By way of organic solvent, there may be mentioned for example lower C₁-C₄ alkanols such as ethanol and isopropanol; glycerol; glycols and glycol ethers such as 2-butoxyethanol, propylene glycol, monomethyl ether of propylene glycol, monoethyl ether and monomethyl ether of diethylene glycol, as well as aromatic alcohols such as benzyl alcohol or phenoxyethanol, similar products and mixtures thereof.

The solvents may be present in proportions preferably of between 1 and 40% by weight approximately relative to the total weight of the dyeing composition, and more preferably still between 5 and 30% by weight approximately.

The pH of the dyeing composition in accordance with the invention is generally between 3 and 12 approximately, preferably between 5 and 11 approximately. It can be adjusted to the desired value by means of acidifying or alkalinizing agents normally used in dyeing keratinous fibres.

Among the acidifying agents, there may be mentioned, by way of example, inorganic or organic acids such as hydrochloric acid, orthophosphoric acid, sulphuric acid, carboxylic acids such as acetic acid, tartaric acid, citric acid, lactic acid and sulphonic acids.

Among the alkalinizing agents, there may be mentioned, by way of example, aqueous ammonia, alkali metal carbonates, alkanolamines such as mono-, di- and triethanolamines as well as their derivatives, sodium or potassium hydroxides and the compounds of the following formula (II):

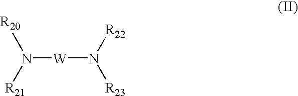

in which W is a propylene residue which is unsubstituted or substituted with a hydroxyl group or a C₁-C₆ alkyl radical; R₂₀, R₂₁, R₂₂ and R₂₃, which are identical or different, represent a hydrogen atom, a C₁-C₆ alkyl or C₁-C₆ hydroxyalkyl radical.

The oxidation dyeing compositions in accordance with the invention may also contain at least one direct dye, in particular for modifying the shades or enriching them with glints.

The dyeing composition in accordance with the invention may also contain various adjuvants conventionally used in hair-dyeing compositions, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, antioxidants, penetrating agents, sequestering agents, perfumes, buffers, dispersing agents, conditioning agents such as for example modified or unmodified, volatile or nonvolatile silicones, film-forming agents, ceramides, preservatives, opacifying agents.

Of course persons skilled in the art will be careful to choose this or these possible additional compounds such that the advantageous properties intrinsically attached to the oxidation dyeing composition in accordance with the invention are not, or not substantially, impaired by the addition(s) envisaged.

The dyeing composition according to the invention may be provided in various forms, such as in the form of liquids, creams, gels, or in any other form appropriate for dyeing keratinous fibres, and in particular human hair.

Another subject of the invention is a method of oxidation dyeing keratinous fibres, and in particular human keratinous fibres such as hair, using the dyeing composition as defined above.

According to this method, at least one dyeing composition as defined above is applied to the fibres, the colour being developed at acidic, neutral or alkaline pH with the aid of an oxidizing agent which is added just at the time of use to the dyeing composition or which is present in an oxidizing composition applied simultaneously or sequentially.

According to a preferred embodiment of the dyeing method of the invention, the dyeing composition described above is preferably mixed, at the time of use, with an oxidizing composition containing, in a medium appropriate for dyeing, at least one oxidizing agent present in a sufficient quantity to develop a colour. The mixture obtained is then applied to the keratinous fibres and allowed to act for 3 to 50 minutes approximately, preferably 5 to 30 minutes approximately, after which they are rinsed, washed with shampoo, rinsed again and dried.

The oxidizing agent may be chosen from the oxidizing agents conventionally used for the oxidation dyeing of keratinous fibres, and among which there may be mentioned hydrogen peroxide, urea peroxide, alkali metal bromates, persalts such as perborates and persulphates, and enzymes such as peroxidases, laccases, tyrosinases and oxidoreductases among which there may be mentioned in particular pyranose oxidases, glucose oxidases, glycerol oxidases, lactate oxidases, pyruvate oxidases and uricases.

The pH of the oxidizing composition containing the oxidizing agent as defined above is such that after mixing with the dyeing composition, the pH of the resulting composition applied to the keratinous fibres preferably varies between 3 and 12 approximately, and more preferably still between 5 and 11. It is adjusted to the desired value by means of acidifying or alkalinizing agents normally used in dyeing keratinous fibres and as defined above.

The oxidizing composition as defined above may also contain various adjuvants conventionally used in hair-dyeing compositions and as defined above.

The composition which is finally applied to the keratinous fibres may be provided in various forms, such as in the form of liquids, creams, gels, or in any other form appropriate for dyeing keratinous fibres, and in particular human hair.

The subject of the invention is finally a multicompartment device or dyeing <<kit>> or any other multicompartment packaging system in which a first compartment contains the dyeing composition as defined above and a second compartment contains the oxidizing composition as defined above. These devices may be equipped with a means which makes it possible to deliver the desired mixture to the hair, such as the devices described in Patent FR-2,586,913 in the name of the applicant.

The following examples are intended to illustrate the invention without however limiting the scope thereof.

### EXAMPLES OF DYEING

### Examples 1 to 3 Of Dyeing in Alkaline Medium

The following dyeing compositions were prepared (contents in grams):

| EXAMPLE | 1 | 2 | 3 |
|---|---|---|---|
| 5-Methylpyrazolo[1,5-a]pyrimidine-3,7-diamine | 5.25 | — | — |
| (oxidation base) | | | |
| 3-Amino-5-methyl-7-imidazolylpropylamino- | — | 7.57 | — |
| pyrazolo[1,5-a]pyrimidine · 2HCl (oxidation base) | | | |
| Pyrazolo[1,5-a]pyrimidine-3,7-diamine | — | — | 1.48 |
| (oxidation base) | | | |
| (2-Hydroxy-4-aminophenyl)carbamic acid ethyl | 4.37 | 4.37 | 1.31 |
| ester | | | |
| (compound of formula (I)) | | | |
| Common dye carrier | (*) | (*) | (*) |
| Dermineralized water qs | 100 g | 100 g | 100 g |

(*) Common dye carrier:

(*) Common dye carrier:

| | | |
|---|---|---|
| Polyglycerolated oleyl alcohol | 4.0 | g |
| containing 2 mol of glycerol | | |
| Polyglycerolated oleyl alcohol | 5.69 | g AS |
| containing 4 mol of glycerol, containing | | |
| 78% of active substances (As) | | |
| Oleic acid | 3.0 | g |
| Oleylamine containing 2 mol of ethylene | 7.0 | g |
| oxide, sold under the trade name | | |
| ETHOMEEN O12 by the company AKZO | | |
| Diethylaminopropyl laurylamino | 3.0 | g AS |
| succinamate, sodium salt, containing 55% AS | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9.0 | g |
| Sodium metabisulphite as an aqueous | 0.455 | g AS |
| solution containing 35% AS | | |
| Ammonium acetate | 0.8 | g |

| | |
|---|---|
| Antioxidant, sequestrant | qs |
| Perfume, preservative | qs |

| | | |
|---|---|---|
| Aqueous ammonia containing 20% NH₃ | 10.2 | g |

At the time of use, each dyeing composition was mixed with an equal quantity of an oxidizing composition consisting of a solution of hydrogen peroxide at 20 volumes (6% by weight) and having a pH of about 3.

Each mixture obtained had a pH of about 9.5 and was applied for 30 minutes to locks of natural grey hair which was 90% white. The locks of hair were then rinsed, washed with a standard shampoo and then dried.

The locks of hair were dyed in the shades which are presented in the table below:

| EXAMPLE | SHADE ON NATURAL HAIR |
|---|---|
| 1 | Slightly coppery red |
| 2 | Slightly golden deeply iridescent |
| 3 | Golden coppery |

1. Composition for the oxidation dyeing of keratinous fibres, characterized in that it contains, in a medium appropriate for dyeing:
at least one oxidation base chosen from the pyrazolopyrimidines;
and at least one coupler chosen from the compounds of the following formula (I), and their addition salts with an acid: 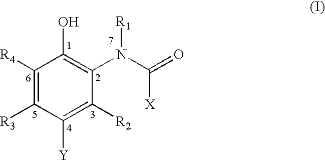
in which:
R₁ represents a hydrogen atom; a radical comprising from 1 to 15 carbon atoms, linear or branched (it being possible for the branch(es) to form one or more carbon-containing rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; it being understood that the said SO₂ group is not directly linked to the nitrogen atom at the 7-position carrying the radical R₁; the said radical R₁ not comprising a peroxide bond or diazo, nitro or nitroso radicals;
X represents a group OR₅ or NR₆R₇;
R₅ represents a radical comprising from 1 to 20 carbon atoms, linear or branched (it being possible for the branch(es) to form one or more carbon-containing rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R₅ not comprising a peroxide bond or diazo, nitro or nitroso radicals;
R₆ and R₇, which are identical or different, represent a hydrogen atom; a radical comprising from 1 to 20 carbon atoms, linear or branched (it being possible for the branch(es) to then form one or more rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radicals R₆ and R₇ not comprising a peroxide bond or diazo, nitro or nitroso radicals;
the radicals R₆ and R₇ may, in addition, be linked in order to form a saturated or unsaturated ring comprising from 5 to 7 members, consisting of carbon, nitrogen, oxygen, sulphur and/or the group C═O, each member being unsubstituted or substituted with 1 or 2 radicals R, which are identical or different, R being a C₁-C₆ alkyl radical, linear or branched (it being possible for the branch(es) to then form one or more rings comprising from 3 to 6 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R not comprising a peroxide bond or diazo, nitro or nitroso radicals;
the radicals R₁ and R₅ may, in addition, be linked in order to form a saturated or unsaturated ring comprising from 5 to 7 members, consisting of carbon, nitrogen, oxygen, sulphur and/or the group C═O, each member being unsubstituted or substituted with 1 or 2 radicals R, which are identical or different, R being a C₁-C₆ alkyl radical, linear or branched (it being possible for the branch(es) to then form one or more rings comprising from 3 to 6 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R not comprising a peroxide bond or diazo, nitro or nitroso radicals;
the radicals R₁ and R₆ may, in addition, be linked in order to form a saturated or unsaturated ring comprising from 5 to 7 members, consisting of carbon, nitrogen, oxygen, sulphur and/or the group C═O, each member being unsubstituted or substituted with 1 or 2 radicals R, which are identical or different, R being a C₁-C₆ alkyl radical, linear or branched (it being possible for the branch(es) to then form one or more rings comprising from 3 to 6 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radical R not comprising a peroxide bond or diazo, nitro or nitroso radicals;
R₂, R₃ and R₄, which are identical or different, represent a hydrogen or halogen atom; a radical comprising from 1 to 20 carbon atoms, linear or branched (it being possible for the branch(es) to then form one or more rings comprising from 3 to 7 members), which may contain one or more double bonds and/or one or more triple bonds (the said double bonds possibly leading to aromatic groups), in which one or more carbon atoms may be replaced with an oxygen, nitrogen or sulphur atom or with an SO₂ group, and in which the carbon atoms may, independently of each other, be substituted with one or more halogen atoms; the said radicals R₂, R₃ and R₄ not comprising a peroxide bond or diazo, nitro or nitroso radicals; it being understood that R₄ cannot represent a hydroxyl, thio or amino radical or a sulphonylamino group which is substituted or unsubstituted; and it being understood that the radicals R₂, R₃ and R₄ cannot be linked to the benzene ring in formula (I) by an —NH—NH— bond;
the radicals R₁ and R₂ may, in addition, be linked so as to form a saturated ring comprising from 5 to 7 members, consisting of carbon, nitrogen, oxygen, sulphur and/or the group C═O, each member being unsubstituted or substituted with 1 or 2 radicals R, which are identical or different, R having the same meanings as those indicated above; the said radical R not comprising a peroxide bond or diazo, nitro or nitroso radicals;
the radicals R₂ and R₅ may also be linked so as to form a saturated ring comprising from 5 to 7 members, consisting of carbon, nitrogen, oxygen, sulphur and/or the group C═O, each member being unsubstituted or substituted with 1 or 2 radicals R, which are identical or different, R having the same meanings as those indicated above; the said radical R not comprising a peroxide bond or diazo, nitro or nitroso radicals;
the radicals R₂ and R₆ may also be linked so as to form a saturated ring comprising from 5 to 7 members, consisting of carbon, nitrogen, oxygen, sulphur and/or the group C═O, each member being unsubstituted or substituted with 1 or 2 radicals R, which are identical or different, R having the same meanings as those indicated above; the said radical R not comprising a peroxide bond or diazo, nitro or nitroso radicals;
Y represents a hydrogen or halogen atom; a group —OR₈, —SR₈, or —NH—SO₂R₈ in which R₈ represents a C₁-C₈ alkyl radical, linear or branched (it being possible for the branch(es) to then form one or more rings comprising from 3 to 6 members), unsubstituted or substituted with one or more radicals chosen from the group consisting of a halogen atom, a hydroxyl, C₁-C₄ alkoxy, amino and amino(C₁-C₄ alkyl) radical; a phenyl radical, unsubstituted or substituted with one or two radicals chosen from the group consisting of a C₁-C₄ alkyl, trifluoromethyl, carboxyl, C₁-C₄ alkoxycarbonyl, halogen, hydroxyl, C₁-C₄ alkoxy, amino and amino(C₁-C₄ alkyl) radical; or a benzyl radical; it being understood that when R₂ represents a hydroxyl radical, then Y cannot represent a group —NH—SO₂R₈;
it being understood that:
when R₂ and R₄ simultaneously represent a hydrogen atom and R₃ represents a substituted or unsubstituted C-C₄ alkyl radical, then X cannot represent a group OR₅ in which R₅ represents a substituted or unsubstituted C₁-C₄ alkyl radical;
when R₂ and R₄ simultaneously represent a hydrogen atom and R₃ represents a substituted or unsubstituted C₁-C₄ alkyl radical, then X cannot represent a group NR₆R₇;
when R₂, R₄ and Y simultaneously represent a hydrogen atom, and X represents a group NR₆R₇ in which R₆ and R₇ simultaneously represent a hydrogen atom, then R₃ is different from a fluorine, chlorine or bromine atom;
when X represents a group NR₆R₇ and R₁ and R₆, which are identical or different, represent a hydrogen atom or a C₁-C₄ alkyl radical or a group —CH₂CONH₂ or —CH₂CON(CH₂CH₃)₂ and R₂ represents a hydrogen atom or a C₁-C₄ alkoxy radical, and R₄ represents an amino radical which is mono- or disubstituted with a C₁-C₄ alkyl or an aryl radical, and R₇ represents a hydrogen atom or a C₁-C₄ alkyl radical or a substituted or unsubstituted aromatic group or a substituted or unsubstituted heteroaromatic group and Y represents a hydrogen or halogen atom or a C₁-C₄ alkoxy group, then R₃ is different from a hydrogen or halogen atom, and from a hydroxyl, amino, C₁-C₄ alkyl, C₁-C₄ alkoxy or aryl group;
when X represents a group NR₆R₇ and R₁ and R₆, which are identical or different, represent a hydrogen atom, a C₁-C₄ alkyl or a group —CH₂CONH₂ or —CH₂CON(CH₂CH₃)₂, and R₄ represents a hydrogen or halogen atom or a C₁-C₄ alkyl, C₁-C₄ alkoxy, or aryl radical, and R₇ represents a hydrogen atom, a C₁-C₄ alkyl radical or a substituted or unsubstituted aromatic group, or a substituted or unsubstituted heteroaromatic group and Y represents a C₁-C₄ alkoxy radical, then R₂ and R₃ are different from a hydrogen or halogen atom, and from a hydroxyl, amino, C₁-C₄ alkyl, C₁-C₄ alkoxy or aryl group.

2. Composition according to claim 1, characterized in that the pyrazolopyrimidines are chosen from pyrazolo[1,5-a]pyrimidine-3,7-diamine; 2-methylpyrazolo[1,5-a]pyrimidine-3,7-diamine; 5-methylpyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)-(2-hydroxyethyl)amino]ethanol; 2-[(7-aminopyrazolo-[1,5-a]pyrimidin-3-yl)-(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine; 3-amino-5-methyl-7-imidazolylpropylaminopyrazolo[1,5-a]pyrimidine, 3-amino-5,6-dimethyl-7-imidazolylpropylaminopyrazolo[1,5-a]pyrimidine; pyrazolo[1,5-a]pyrimidin-3-ylamine; 5,7-dimethylpyrazolo[1,5-a]pyrimidin-3-ylamine; 5,6,7-trimethylpyrazolo[1,5-a]pyrimidin-3-ylamine; 7-methylpyrazolo[1,5-a]pyrimidin-3-ylamine; 5-methylpyrazolo[1,5-a]pyrimidin-3-ylamine; 7-methoxypyrazolo[1,5-a]pyrimidin-3-ylamine; 5-methoxypyrazolo[1,5-a]pyrimidin-3-ylamine; 7-methoxy-5-methylpyrazolo[1,5-a]pyrimidin-3-ylamine; 2,5,6,7-tetramethylpyrazolo[1,5-a]pyrimidin-3-ylamine; 2-methoxy-5,7-dimethylpyrazolo[1,5-a]pyrimidin-3-ylamine; 5,7-di-tert-butylpyrazolo[1,5-a]pyrimidin-3-ylamine; 5,7-di-trifluoromethylpyrazolo-[1,5-a]pyrimidin-3-ylamine; 2,6-dimethylpyrazolo-[1,5-a]pyrimidin-3-ylamine; 2-chloro-5,7-dimethylpyrazolo[1,5-a]pyrimidin-3-ylamine; 7-methyl-2-tert-butyl-5-trifluoromethylpyrazolo[1,5-a]pyrimidin-3-ylamine; 3-amino-7-imidazolylpropylaminopyrazolo-[1,5-a]pyrimidine; 3-amino-7-dimethylaminoethylaminopyrazolo[1,5-a]pyrimidine; 7-methoxy-2,5-dimethylpyrazolo[1,5-a]pyrimidin-3-ylamino; 7-methoxy-2-methylpyrazolo[1,5-a]pyrimidine-3,5-diamine; 7-methoxy-2-methylpyrazolo[1,5-a]pyrimidin-3-ylamine; 7-methoxy-2,5,6-trimethylpyrazolo[1,5-a]pyrimidin-3-ylamine; 7-methoxy-2,6-dimethylpyrazolo[1,5-a]-pyrimidine-3,5-diamine; 7-methoxy-2,6-dimethylpyrazolo-[1,5-a]pyrimidin-3-ylamine; 2-(3-amino-2,5-dimethylpyrazolo[1,5-a]pyrimidin-7-yloxy)ethanol; 2-(3,5-diamino-2-methylpyrazolo[1,5-a]pyrimidin-7-yloxy)ethanol; 2-(3-amino-2-methylpyrazolo[1,5-a]-pyrimidin-7-yloxy)ethanol; 2-(3-amino-2,5,6-trimethylpyrazolo[1,5-a]pyrimidin-7-yloxy)ethanol; 2-(3,5-diamino-2,6-dimethylpyrazolo[1,5-a]pyrimidin-7-yloxy)ethanol; 2-(3-amino-2,6-dimethylpyrazolo-[1,5-a]pyrimidin-7-yloxy)ethanol; 5,6-dimethylpyrazolo[1,5-a]pyrimidin-3-ylamine; 7-methoxy-5,6-dimethylpyrazolo[1,5-a]pyrimidin-3-ylamine; 5,7-dimethylpyrazolo[1,5-a]pyridimin-3-ylamine; pyrazolo[1,5-a]pyrimidine-3,5-diamine; 6-methylpyrazolo[1,5-a]pyrimidine-3,5,7-triamine; 6,7-dimethylpyrazolo[1,5-a]pyrimidine-3,5-diamine; 6-methylpyrazolo[1,5-a]pyrimidine-3,5-diamine; 7-methoxy-6-methylpyrazolo[1,5-a]pyrimidine-3,5-diamine; 3-amino-7-methylpyrazolo[1,5-a]pyrimidin-5-ol; 3,7-diamino-7-methylpyrazolo[1,5-a]pyrimidin-5-ol; 3-amino-6,7-dimethylpyrazolo[1,5-a]pyrimidin- 5-ol; 3,7-diamino-6-methylpyrazolo[1,5-a]pyrimidin-5-ol; 2-methoxy-5-methylpyrazolo[1,5-a]pyrimidine-3,7-diamine; 2,7-dimethoxy-5-methylpyrazolo[1,5-a]-pyrimidin-3-ylamine; 2-methoxy-5-methylpyrazolo[1,5-a]-pyrimidin-3-ylamine; 2-methoxypyrazolo[1,5-a]pyrimidin-3-ylamine; 2-methoxy-7-methylpyrazolo[1,5-a]pyrimidin-3-ylamine; 2-methoxypyrazolo[1,5-a]pyrimidine-3,7-diamine; 2,7-dimethoxypyrazolo[1,5-a]pyrimidin-3-ylamine; and their addition salts with an acid.

3. Composition according to claim 2, characterized in that the pyrazolopyrimidines are chosen from 5-methylpyrazolo[1,5-a]pyrimidine-3,7-diamine; pyrazolo[1,5-a]pyrimidine-3,7-diamine; 2-methylpyrazolo[1,5-a]pyrimidine-3,7-diamine; 3-amino-5-methyl-7-imidazolylpropylaminopyrazolo[1,5-a]-pyrimidine; 3-amino-5,6-dimethyl-7-imidazolylpropylaminopyrazolo[1,5-a]pyrimidine; 2-methoxy-5,7-dimethylpyrazolo[1,5-a]pyrimidin-3-ylamine; 3-amino-7-imidazolylpropylaminopyrazolo-[1,5-a]pyrimidine; and their addition salts with an acid.

4. Composition according to any one of the preceding claims, characterized in that the pyrazolopyrimidines represent from 0.0005 to 12% by weight of the total weight of the dyeing composition.

5. Composition according to any one of the preceding claims, characterized in that in the compounds of formula (I), R₁ denotes a hydrogen atom; a group A₁ consisting of a linear or branched C₁-C₈ alkyl radical which may carry one or two double bonds or one triple bond, which may be unsubstituted or substituted with a group chosen from the groups A₂, A₄ and A₅ as defined below, which may be unsubstituted or substituted with one or two groups, which are identical or different, chosen from the N-(C₁-C₃)alkylamino, N-(C₁-C₃)alkyl-N-(C₁-C₃)alkylamino, (C₁-C₆)alkoxy, oxo, alkoxycarbonyl, acyloxy, amide, acylamino, ureyl, sulphoxy, sulphonyl, sulphonamido, sulphonylamino, bromo, cyano and carboxyl groups, and may be unsubstituted or substituted with one or more hydroxyl, fluoro or chloro groups; a group A₂ consisting of an aromatic group of the phenyl or naphthyl type, which may be unsubstituted or substituted with one to three groups, which are identical or different, chosen from the methyl, trifluoromethyl, ethyl, isopropyl, butyl, pentyl, fluoro, chloro, bromo, methoxy, trifluoromethoxy, ethoxy, propyloxy, acetyloxy, acetyl and cyano groups; a group A₃ consisting of heteroaromatic groups chosen from the furanyl, thiophenyl, pyrrolyl, imidazolyl, thiazolyl, oxazolyl, 1,2,3-triazolyl, 1,2,4-triazolyl, isoxazolyl, isothiazolyl, pyrazolyl, pyrazolotriazolyl, pyrazoloimidazolyl, pyrrolotriazolyl, pyrazolopyrimidyl, pyrazolopyridyl, pyridyl, pyrimidyl, benzoimidazolyl, benzoxazolyl, benzothiazolyl, indolyl, indolidinyl, isoindolyl, indazolyl, benzotriazolyl, quinolinyl, benzoimidazolyl and benzopyrimidyl groups, the said groups being unsubstituted or substituted with 1 to 3 radicals chosen from a linear or branched C₁-C₄ alkyl radical, a C₁-C₄ monohydroxyalkyl radical, a C₂-C₄ polyhydroxyalkyl radical, a carboxyl radical, an alkoxycarbonyl radical, a halogen atom, an amido radical, an amino radical and a hydroxyl radical; a group A₄ consisting of a C₃-C₇ cycloalkyl radical, a norbornyl radical, carrying or otherwise a double bond and unsubstituted or substituted with 1 or 2 radicals chosen from a linear or branched C₁-C₄ alkyl radical, a C₁-C₄ monohydroxyalkyl radical, a C₂-C₄ polyhydroxyalkyl radical, a carboxyl radical, an alkoxycarbonyl radical, a halogen atom, an amido radical, an amino radical and a hydroxyl radical; a group A₅ consisting of a heterocycle chosen from the dihydrofuranyl, tetrahydrofuranyl, butyrolactonyl, dihydrothiophenyl, tetrahydrothiophenyl, tetrahydrothiophenonyl, iminothiolane, dihydropyrrolyl, pyrrolidinyl, pyrrolidinonyl, imidazolidinonyl, imidazolidinethionyl, oxazolidinyl, oxazolidinonyl, oxazolanethione, thiazolidinyl, isothiazolonyl, mercaptothiazolinyl, pyrazolidinonyl, iminothiolane, dioxolanyl, pentalactone, dioxanyl, dihydropyridinyl, piperidinyl, pentalactam, morpholinyl, pyrazoli(di)nyl, pyrimi(di)nyl, pyrazinyl, piperazinyl and azepinyl rings; the said groups being optionally separated from the nitrogen present at the 7-position to which the radical R₁ is attached by a group —(CO)—.

6. Composition according to claim 5, characterized in that R₁ represents a hydrogen atom; a methyl, ethyl, isopropyl, allyl, phenyl, benzyl, fluorobenzyl, difluorobenzyl, trifluorobenzyl, chlorobenzyl, bromobenzyl, methoxybenzyl, dimethoxybenzyl, (trifluoromethoxy)benzyl, 3,4-methylenedioxybenzyl, 6-chloropiperonyl, 4-methylthiobenzyl, 4-methylsulphonylbenzyl, 4-acetylaminobenzyl, 4-carboxybenzyl, 1-naphthomethyl or 2-naphthomethyl radical; or a 2-hydroxyethyl, 2-methoxyethyl or 2-ethoxyethyl group.

7. Composition according to any one of the preceding claims, characterized in that in the compounds of formula (I), R₅ denotes a group A₁, A₂, A₃, A₄ or A₅ as defined in claim 2.

8. Composition according to claim 7, characterized in that R₅ denotes a methyl, ethyl, propyl, isopropyl, butyl, isobutyl, tert-butyl, pentyl, isopentyl, neopentyl, hexyl; cyclopropyl, cyclobutyl, cyclopentyl, cyclohexyl, vinyl, 1-methylvinyl, 2-methylvinyl, 2,2-dimethylvinyl, allyl, propargyl, chloromethyl, 2-chloroethyl, 2-hydroxyethyl, 2-methoxyethyl, 4-chlorobutyl; phenyl, 4-methylphenyl, 4-ethylphenyl, 4-(trifluoromethyl)phenyl, 4-hydroxyphenyl, 4-methoxyphenyl, 4-ethoxyphenyl, 4-acetoxyphenyl, 4-aminophenyl, 4-fluorophenyl, 4-chlorophenyl, 4-bromophenyl, naphth-1-yl, naphth-2-yl; or benzyl radical.

9. Composition according to any one of the preceding claims, characterized in that in the compounds of formula (I), R₆ and R₇, which are identical or different, denote a group A₁, A₂, A₃, A₄ or As as defined in claim 5.

10. Composition according to claim 9, characterized in that R₆ and R₇, which are identical or different, denote a hydrogen atom or a methyl, ethyl, propyl, isopropyl, butyl, isobutyl, tert-butyl, pentyl, isopentyl, neopentyl, hexyl; cyclopentyl, cyclohexyl, allyl, propargyl, 2-chloroethyl, 2-hydroxyethyl, 2-methoxyethyl, 3-chloropropyl, 3-hydroxypropyl, 2,3-dihydroxypropyl, carboxymethyl, phenyl, fluorophenyl, (trifluoromethyl)phenyl, chlorophenyl, bromophenyl, methylphenyl, 4-acetylphenyl, methoxyphenyl, (trifluoromethoxy)phenyl, naphth-1-yl, benzyl, phenethyl or pyrid-3-yl radical.

11. Composition according to any one of the preceding claims, characterized in that in the compounds of formula (I), R₂ and R₃, which are identical or different, denote a hydrogen or halogen atom; a hydroxyl or amino group; a group A₁, A₄ or A₅ as defined in claim 5; a group A₁, A₂, A₃, A₄ or A₅ as defined in claim 5 and separated from the phenolic nucleus in formula (I) by an oxygen atom or by a group —NH—, —Nalkyl(C₁-C₃)—, —O(CO)—, —NH(CO)—, -Nalkyl(C₁-C₃)(CO)—, —NH[C═NH]—, —NH(CO)NH—, —NH(CO)Nalkyl(C₁-C₃)—, —NH(CO)O—, —NHSO₂—, —NHSO₂NH— or —NHSO₂Nalkyl(C₁-C₃)—.

12. Composition according to claim 11, characterized in that R₂ represents a hydrogen or chlorine atom; a methyl, hydroxymethyl, methoxymethyl, 2-hydroxyethyl, aminomethyl, methylaminomethyl, hydroxyl, methoxy, acetoxy, amino, methylamino or 2-hydroxyethylamino radical; a group —NH(CO)R₉ in which R₉ represents one of the radicals listed in the group (G1) consisting of a methyl, ethyl, propyl, isopropyl, butyl, isobutyl, tert-butyl, pentyl, isopentyl, neopentyl, hexyl; cyclopropyl, cyclobutyl, cyclopentyl, cyclopentylmethyl, 3-cyclopentylpropyl, cyclohexyl, 2-cyclohexylethyl, norborn-2-yl, vinyl, 1-methylvinyl, 2-methylvinyl, 2,2-dimethylvinyl, allyl, 3-butenyl; phenyl, methylphenyl, dimethylphenyl, 2,4,6-trimethylphenyl, 4-ethylphenyl, (trifluoromethyl)phenyl, hydroxyphenyl, methoxyphenyl, ethoxyphenyl, acetoxyphenyl, (trifluoromethoxy)phenyl, aminophenyl, 4-dimethylaminophenyl, fluorophenyl, difluorophenyl, fluoro(trifluoromethyl)phenyl, chlorophenyl, dichlorophenyl, bromophenyl, naphth-1-yl, naphth-2-yl, (2-methoxy)naphth-1-yl, benzyl, 4′-methoxybenzyl, 2′,5′-dimethoxybenzyl, 3′,4′-dimethoxybenzyl, 4′-fluorobenzyl, 4′-chlorobenzyl, phenethyl, 2-phenylvinyl, (1-naphthyl)methyl, (2-naphthyl)methyl; tetrahydrofuran-2-yl, furan-2-yl, 5-methyl-2-(trifluoromethyl)furan-3-yl, 2-methyl-5-phenylfuran-3-yl, thiophen-2-yl, (thiophen-2-yl)methyl, 3-chlorothiophen-2-yl, 2,5-dichlorothiophen-3-yl, benzothiophen-2-yl, 3-chlorobenzothiophen-2-yl, isoxazol-5-yl, 5-methylisoxazol-3-yl, 3,5-dimethylisoxazol-4-yl, 1,3-dimethylpyrazol-5-yl, 1-ethyl-3-methylpyrazol-5-yl, 1-tert-butyl-3-methylpyrazol-5-yl, 3-tert-butyl-1-methylpyrazol-5-yl, 4-bromo-1-ethyl-3-methylpyrazol-5-yl, indol-3-ylcarboxyl, pyridinyl, chloropyridinyl, dichloropyridinyl, 5-(bromo)pyridin-3-yl, piperazin-2-yl, quinoxal-2-yl; fluoromethyl, difluoromethyl, trifluoromethyl, 1,1,2,2-tetrafluoroethyl, pentafluoroethyl, heptafluoropropyl, 1,1,2,2,3,3,4,4-octafluorobutyl, nonafluorobutyl, chloromethyl, chloroethyl, 1,1-dimethyl-2-chloroethyl, 1,2-dichloroethyl, 1-chloropropyl, 3-chloropropyl, 4-chlorobutyl, hydroxymethyl, methoxymethyl, phenoxymethyl, (4-chlorophenoxy)methyl, benzyloxymethyl, acetoxymethyl, 1,2-dihydroxyethyl, 1-phenoxyethyl, 1-acetoxyethyl, 2-(2-carboxyethoxy)ethyl, 1-phenoxyethyl, 1-acetoxyethyl, methoxycarbonyl, ethoxycarbonyl, (methoxycarbonyl)methyl, 2-carboxyethyl, 2-(methoxycarbonyl)ethyl, 2-carboxycyclopropyl, 2-carboxycyclohexane; methoxy, ethoxy, propoxy, isopropoxy, butoxy, isobutoxy, pentoxy, neopentoxy, hexyloxy, cyclopentyloxy, cyclohexyloxy, vinyloxy, allyloxy, propargyloxy, chloromethoxy, 1-chloroethoxy, 2-methoxyethoxy, 4-chlorobutoxy, phenoxy, 4-methylphenoxy, 4-fluorophenoxy, 4-bromophenoxy, 4-chlorophenoxy, 4-methoxyphenoxy, naphth-2-yloxy, benzyloxy; amino, methylamino, ethylamino, propylamino, isopropylamino, butylamino, cyclohexylamino, allylamino, 2-chloroethylamino, 3-chloropropylamino, carboxymethylamino, phenylamino, fluorophenylamino, (trifluoromethyl)phenylamino, chlorophenylamino, bromophenylamino, 4-acetylphenylamino, methoxyphenylamino, (trifluoromethoxy)phenylamino, naphth-1-ylamino, benzylamino, phenethylamino, pyrid-3-ylamino, dimethylamino, 1-pyrrolidinyl and 4-morpholinyl radical; a group —NHSO₂R₁₀ in which R₁₀ represents one of the radicals listed in the group (G2) consisting of the methyl, trifluoromethyl, ethyl, 2-chloroethyl, propyl, 3-chloropropyl, isopropyl, butyl, thiophen-2-yl, hydroxyl, ethoxy and dimethylamino radicals.

13. Composition according to any one of the preceding claims, characterized in that in the compounds of formula (I), R₃ represents a hydrogen or chlorine atom; a methyl, ethyl, hydroxymethyl, 2-hydroxyethyl, methoxymethyl, aminomethyl, methylaminomethyl, hydroxyl, methoxy, acetoxy, amino, methylamino, 2-hydroxyethylamino, N-piperidino or N-morpholino radical; a group -NH(CO)R₁₂ in which R₁₂ represents one of the radicals listed in the group (G1) consisting of a methyl, ethyl, propyl, isopropyl, butyl, isobutyl, tert-butyl, pentyl, isopentyl, neopentyl, hexyl; cyclopropyl, cyclobutyl, cyclopentyl, cyclopentylmethyl, 3-cyclopentylpropyl, cyclohexyl, 2-cyclohexylethyl, norborn-2-yl, vinyl, 1-methylvinyl, 2-methylvinyl, 2,2-dimethylvinyl, allyl, 3-butenyl; phenyl, methylphenyl, dimethylphenyl, 2,4,6-trimethylphenyl, 4-ethylphenyl, (trifluoromethyl)phenyl, hydroxyphenyl, methoxyphenyl, ethoxyphenyl, acetoxyphenyl, (trifluoromethoxy)phenyl, aminophenyl, 4-dimethylaminophenyl, fluorophenyl, difluorophenyl, fluoro(trifluoromethyl)phenyl, chlorophenyl, dichlorophenyl, bromophenyl, naphth-1-yl, naphth-2-yl, (2-methoxy)naphth-1-yl, benzyl, 4!-methoxybenzyl, 2′,5′-dimethoxybenzyl, 3′,4′-dimethoxybenzyl, 4′-fluorobenzyl, 4′-chlorobenzyl, phenethyl, 2-phenylvinyl, (1-naphthyl)methyl, (2-naphthyl)methyl; tetrahydrofuran-2-yl, furan-2-yl, 5-methyl-2-(trifluoromethyl)furan-3-yl, 2-methyl-5-phenylfuran -3-yl, thiophen-2-yl, (thiophen-2-yl)methyl, 3-chlorothiophen -2-yl, 2,5-dichlorothiophen-3-yl, benzothiophen -2-yl, 3-chlorobenzothiophen-2-yl, isoxazol -5-yl, 5-methylisoxazol-3-yl, 3,5-dimethylisoxazol-4-yl, 1,3-dimethylpyrazol-5-yl, 1-ethyl-3-methylpyrazol-5-yl, 1-tert-butyl-3-methylpyrazol-5-yl, 3-tert-butyl -1-methylpyrazol-5-yl, 4-bromo-1-ethyl-3-methylpyrazol -5-yl, indol-3-ylcarboxyl, pyridinyl, chloropyridinyl, dichloropyridinyl, 5-(bromo)pyridin-3-yl, piperazin -2-yl, quinoxal-2-yl; fluoromethyl, difluoromethyl, trifluoromethyl, 1,1,2,2-tetrafluoroethyl, pentafluoroethyl, heptafluoropropyl, 1,1,2,2,3,3,4,4-octafluorobutyl, nonafluorobutyl, chloromethyl, chloroethyl, 1,1-dimethyl-2-chloroethyl, 1,2-dichloroethyl, 1-chloropropyl, 3-chloropropyl, 4-chlorobutyl, hydroxymethyl, methoxymethyl, phenoxymethyl, (4-chlorophenoxy)methyl, benzyloxymethyl, acetoxymethyl, 1,2-dihydroxyethyl, 1-phenoxyethyl, 1-acetoxyethyl, 2-(2-carboxyethoxy)ethyl, 1-phenoxyethyl, 1-acetoxyethyl, methoxycarbonyl, ethoxycarbonyl, (methoxycarbonyl)methyl, 2-carboxyethyl, 2-(methoxycarbonyl)ethyl, 2-carboxycyclopropyl, 2-carboxycyclohexane; methoxy, ethoxy, propoxy, isopropoxy, butoxy, isobutoxy, pentoxy, neopentoxy, hexyloxy, cyclopentyloxy, cyclohexyloxy, vinyloxy, allyloxy, propargyloxy, chloromethoxy, 1-chloroethoxy, 2-methoxyethoxy, 4-chlorobutoxy, phenoxy, 4-methylphenoxy, 4-fluorophenoxy, 4-bromophenoxy, 4-chlorophenoxy, 4-methoxyphenoxy, naphth-2-yloxy, benzyloxy; amino, methylamino, ethylamino, propylamino, isopropylamino, butylamino, cyclohexylamino, allylamino, 2-chloroethylamino, 3-chloropropylamino, carboxymethylamino, phenylamino, fluorophenylamino, (trifluoromethyl)phenylamino, chlorophenylamino, bromophenylamino, 4-acetylphenylamino, methoxyphenylamino, (trifluoromethoxy)phenylamino, naphth-1-ylamino, benzylamino, phenethylamino, pyrid-3-ylamino, dimethylamino, 1-pyrrolidinyl and 4-morpholinyl radical; or a group —NHSO₂R₁₃ in which R₁₃ represents one of the radicals listed in the group (G2) consisting of the methyl, trifluoromethyl, ethyl, 2-chloroethyl, propyl, 3-chloropropyl, isopropyl, butyl, thiophen-2-yl, hydroxyl, ethoxy and dimethylamino radicals.

14. Composition according to any one of the preceding claims, characterized in that in the compounds of formula (I), R₄ represents a hydrogen or halogen atom; a group A₁, A₄ or As as defined in claim 5; a group A₁, A₂, A₃, A₄ or A₅ as defined in claim 5 and separated from the phenolic nucleus of the compounds of formula (I) by an oxygen or sulphur atom or by a group —NH—, -Nalkyl(C₁-C₃)—, —O(CO)—, —NH(CO)—, -Nalkyl(C₁-C₃)(CO)—, —NH[C═NH]—, —NH(CO)NH—, —NH(CO)Nalkyl(C₁-C₃)— or —NH(CO)O—.

15. Composition according to claim 14, characterized in that R₄ represents a hydrogen, chlorine, fluorine or bromine atom; a methyl, trifluoromethyl, allyl, hydroxymethyl, methoxymethyl, 2-hydroxyethyl, aminomethyl, methylaminomethyl, methoxy, acetoxy or methylamino radical; or a group —NH(CO)R₁₅ in which R₁₅ represents one of the radicals listed in the group (G1) consisting of a methyl, ethyl, propyl, isopropyl, butyl, isobutyl, tert-butyl, pentyl, isopentyl, neopentyl, hexyl; cyclopropyl, cyclobutyl, cyclopentyl, cyclopentylmethyl, 3-cyclopentylpropyl, cyclohexyl, 2-cyclohexylethyl, norborn-2-yl, vinyl, 1-methylvinyl, 2-methylvinyl, 2,2-dimethylvinyl, allyl, 3-butenyl; phenyl, methylphenyl, dimethylphenyl, 2,4,6-trimethylphenyl, 4-ethylphenyl, (trifluoromethyl)phenyl, hydroxyphenyl, methoxyphenyl, ethoxyphenyl, acetoxyphenyl, (trifluoromethoxy)phenyl, aminophenyl, 4-dimethylaminophenyl, fluorophenyl, difluorophenyl, fluoro(trifluoromethyl)phenyl, chlorophenyl, dichlorophenyl, bromophenyl, naphth-1-yl, naphth-2-yl, (2-methoxy)naphth-1-yl, benzyl, 4′-methoxybenzyl, 2′,5′-dimethoxybenzyl, 31,41-dimethoxybenzyl, 4′-fluorobenzyl, 4′-chlorobenzyl, phenethyl, 2-phenylvinyl, (1-naphthyl)methyl, (2-naphthyl)methyl; tetrahydrofuran-2-yl, furan-2-yl, 5-methyl-2-(trifluoromethyl)furan-3-yl, 2-methyl-5-phenylfuran-3-yl, thiophen-2-yl, (thiophen-2-yl)methyl, 3-chlorothiophen-2-yl, 2,5-dichlorothiophen-3-yl, benzothiophen-2-yl, 3-chlorobenzothiophen-2-yl, isoxazol-5-yl, 5-methylisoxazol-3-yl, 3,5-dimethylisoxazol-4-yl, 1,3-dimethylpyrazol-5-yl, 1-ethyl-3-methylpyrazol-5-yl, 1-tert-butyl-3-methylpyrazol-5-yl, 3-tert-butyl-1-methylpyrazol-5-yl, 4-bromo-1-ethyl-3-methylpyrazol-5-yl, indol-3-ylcarboxyl, pyridinyl, chloropyridinyl, dichloropyridinyl, 5-(bromo)pyridin-3-yl, piperazin -2-yl, quinoxal-2-yl; fluoromethyl, difluoromethyl, trifluoromethyl, 1,1,2,2-tetrafluoroethyl, pentafluoroethyl, heptafluoropropyl, 1,1,2,2,3,3,4,4-octafluorobutyl, nonafluorobutyl, chloromethyl, chloroethyl, 1,1-dimethyl-2-chloroethyl, 1,2-dichloroethyl, 1-chloropropyl, 3-chloropropyl, 4-chlorobutyl, hydroxymethyl, methoxymethyl, phenoxymethyl, (4-chlorophenoxy)methyl, benzyloxymethyl, acetoxymethyl, 1,2-dihydroxyethyl, 1-phenoxyethyl, 1-acetoxyethyl, 2-(2-carboxyethoxy)ethyl, 1-phenoxyethyl, 1-acetoxyethyl, methoxycarbonyl, ethoxycarbonyl, (methoxycarbonyl)methyl, 2-carboxyethyl, 2-(methoxycarbonyl)ethyl, 2-carboxycyclopropyl, 2-carboxycyclohexane; methoxy, ethoxy, propoxy, isopropoxy, butoxy, isobutoxy, pentoxy, neopentoxy, hexyloxy, cyclopentyloxy, cyclohexyloxy, vinyloxy, allyloxy, propargyloxy, chloromethoxy, 1-chloroethoxy, 2-methoxyethoxy, 4-chlorobutoxy, phenoxy, 4-methylphenoxy, 4-fluorophenoxy, 4-bromophenoxy, 4-chlorophenoxy, 4-methoxyphenoxy, naphth-2-yloxy, benzyloxy; amino, methylamino, ethylamino, propylamino, isopropylamino, butylamino, cyclohexylamino, allylamino, 2-chloroethylamino, 3-chloropropylamino, carboxymethylamino, phenylamino, fluorophenylamino, (trifluoromethyl)phenylamino, chlorophenylamino, bromophenylamino, 4-acetylphenylamino, methoxyphenylamino, (trifluoromethoxy)phenylamino, naphth-1-ylamino, benzylamino, phenethylamino, pyrid-3-ylamino, dimethylamino, 1-pyrrolidinyl and 4-morpholinyl radical.

16. Composition according to any one of the preceding claims, characterized in that in the compounds of formula (I), Y is chosen from a hydrogen, chlorine, fluorine or bromine atom; a group methoxy, ethoxy, propoxy, benzyloxy, phenoxy, —OCH₂CH₂₀CH₃; —OCH₂CH₂₀CH₃; —OCH₂CH₂N (CH₃)₂; —OCH₂(CO)OH, —OCH₂(CO)OCH₃, —OCH₂(CO)OC₂H₅, —SCH₂CH₂CO₂H, or —NHSO₂CH₃; it being understood that Y cannot represent a group —NHSO₂CH₃ when R₂ represents a hydroxyl radical.

17. Composition according to any one of the preceding claims, characterized in that the compounds of formula (I) are chosen from those in which:
i)
R₁ represents a hydrogen atom;
X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;
R₂ represents a hydroxyl, amino, methylamino, 2-hydroxyethylamino or diethylamino radical; a group —NH(CO)R₁₇ in which R₁₇ represents one of the radicals listed in the group (G4) consisting of a methyl, methoxymethyl, 2-carboxyethyl, methoxy, amino, ethylamino and 1-pyrrolidinyl radical; a methanesulphonylamino, ethanesulphonylamino or dimethylaminosulphonylamino group;
R₃ represents a hydrogen or chlorine atom, or a methyl group;
R₄ represents a hydrogen, chlorine or fluorine atom or a methyl group;
Y represents a hydrogen or chlorine atom; a methoxy or —OCH₂(CO)OCH₃ group;
ii)
R₁ represents a hydrogen atom;
X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;
R₂ represents a hydrogen atom; or a methyl radical;
R₃ represents a hydroxyl, amino, methylamino, 2-hydroxyethylamino, diethylamino, methanesulphonylamino, ethanesulphonylamino or dimethylaminosulphonylamino radical; a group —NH(CO)R₁₈ in which R₁₈ represents one of the radicals listed in the group (G4) defined above;
R₄ represents a hydrogen, chlorine or fluorine atom; or a methyl group;
Y represents a hydrogen or chlorine atom; a methoxy or —OCH₂(CO)OCH₃ group;
iii)
R₁ represents a hydrogen atom;
X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;
R₂ represents a hydrogen atom; or a methyl radical;
R₃ represents a hydrogen or chlorine atom; a methyl radical; or a methoxy or methylamino group;
R₄ represents a group —NH(CO)R₁₉ in which R₁₉ represents one of the radicals listed in the group (G4) defined above;
Y represents a hydrogen or chlorine atom; or a methoxy or —OCH₂(CO)OCH₃ group;
iv)
R₁ represents a hydrogen atom;
X represents a methoxy, ethoxy, propyloxy, isopropyloxy, butyloxy, isobutyloxy, 2-hydroxyethoxy, 2-methoxyethoxy, allyloxy, amino, methylamino, ethylamino, propylamino, isopropylamino, butylamino, isobutylamino, 2-hydroxyethylamino, 2-methoxyethylamino, 3-hydroxypropylamino, 2,3-dihydroxypropylamino, allylamino, dimethylamino, diethylamino, di(2-hydroxyethyl)amino, pyrrolidin-1-yl, 3-hydroxypyrrolidin-1-yl, or 3,4-dihydroxypyrrolidin-1-yl radical;
R₂ represents a hydrogen atom; or a methyl radical;
R₃ represents a hydrogen atom; or a methyl or ethyl group;
R₄ represents a hydrogen, chlorine or fluorine atom;
Y represents a hydrogen or chlorine atom; or a group —OCH₂(CO)OCH₃.

18. Composition according to any one of the preceding claims, characterized in that the compounds of formula (I) are chosen from:
(2-Hydroxyphenyl)carbamic acid methyl ester,
(2-Hydroxyphenyl)carbamic acid ethyl ester,
(2-Hydroxyphenyl)carbamic acid 2-hydroxyethyl ester,
(2-Hydroxyphenyl)carbamic acid isopropyl ester,
1-(2-Hydroxyphenyl)urea,
3-(2-Hydroxyphenyl)-1-methylurea,
3-(2-Hydroxyphenyl)-1-ethylurea,
3-(2-Hydroxyphenyl)-1-(2-hydroxyethyl)urea,
3-(2-Hydroxyphenyl)-1-isopropylurea,
3-(2-Hydroxyphenyl)-1,1-dimethylurea,
3-(2-Hydroxyphenyl)-1,1-diethylurea,
3-(2-Hydroxyphenyl)-1,1-di(2-hydroxyethyl)urea,
(2-Hydroxy-5-chlorophenyl)carbamic acid methyl ester,
(2-Hydroxy-5-chlorophenyl)carbamic acid ethyl ester,
(2-Hydroxy-5-chlorophenyl)carbamic acid 2-hydroxyethyl ester,
(2-Hydroxy-5-chlorophenyl)carbamic acid isopropyl ester,
1-(2-Hydroxy-5-chlorophenyl)urea,
3-(2-Hydroxy-5-chlorophenyl)-1-methylurea,
3-(2-Hydroxy-5-chlorophenyl)-1-ethylurea,
3-(2-Hydroxy-5-chlorophenyl)-1-(2-hydroxyethyl)urea,
3-(2-Hydroxy-5-chlorophenyl)-1-isopropylurea,
3-(2-Hydroxy-5-chlorophenyl)-1,1-dimethylurea,
3-(2-Hydroxy-5-chlorophenyl)-1,1-diethylurea,
3-(2-Hydroxy-5-chlorophenyl)-1,1-di(2-hydroxyethyl)urea,
(2-Hydroxy-5-methoxyphenyl)carbamic acid methyl ester,
(2-Hydroxy-5-methoxyphenyl)carbamic acid ethyl ester,
(2-Hydroxy-5-methoxyphenyl)carbamic acid 2-hydroxyethyl ester,
(2-Hydroxy-5-methoxyphenyl)carbamic acid isopropyl ester,
3-(2-Hydroxy-5-methoxyphenyl)-1-(2-hydroxyethyl)urea,
3-(2-Hydroxy-5-methoxyphenyl)-1,1-di(2-hydroxyethyl)urea,
(2-Hydroxy-6-aminophenyl)carbamic acid methyl ester,
(2-Hydroxy-6-aminophenyl)carbamic acid ethyl ester,
(2-Hydroxy-6-aminophenyl)carbamic acid 2-hydroxyethyl ester,
(2-Hydroxy-6-aminophenyl)carbamic acid isopropyl ester,
1-(2-Hydroxy-6-aminophenyl)urea,
3-(2-Hydroxy-6-aminophenyl)-1-methylurea,
3-(2-Hydroxy-6-aminophenyl)-1-ethylurea,
3-(2-Hydroxy-6-aminophenyl)-1-(2-hydroxyethyl)urea,
3-(2-Hydroxy-6-aminophenyl)-1-isopropylurea,
3-(2-Hydroxy-6-aminophenyl)-1,1-dimethylurea,
3-(2-Hydroxy-6-aminophenyl)-1,1-diethylurea,
3-(2-Hydroxy-6-aminophenyl)-1,1-di(2-hydroxyethyl)urea,
(2-Hydroxy-4-aminophenyl)carbamic acid methyl ester,
(2-Hydroxy-4-aminophenyl)carbamic acid ethyl ester,
(2-Hydroxy-4-aminophenyl)carbamic acid 2-hydroxyethyl ester,
(2-Hydroxy-4-aminophenyl)carbamic acid isopropyl ester,
1-(2-Hydroxy-4-aminophenyl)urea,
3-(2-Hydroxy-4-aminophenyl)-1-methylurea,
3-(2-Hydroxy-4-aminophenyl)-1-ethylurea,
3-(2-Hydroxy-4-aminophenyl)-1-(2-hydroxyethyl)urea,
3-(2-Hydroxy-4-aminophenyl)-1-isopropylurea,
3-(2-Hydroxy-4-aminophenyl)-1,1-dimethylurea,
3-(2-Hydroxy-4-aminophenyl)-1,1-diethylurea,
3-(2-Hydroxy-4-aminophenyl)-1,1-di(2-hydroxyethyl)urea,
Pyrrolidine-1-carboxylic acid (4-amino-2-hydroxyphenyl)amide,
3-Hydroxypyrrolidine-1-carboxylic acid (4-amino-2-hydroxyphenyl)amide,
(2-Hydroxy-4-methylaminophenyl)carbamic acid methyl ester,
(2-Hydroxy-4-methylaminophenyl)carbamic acid ethyl ester,
(2-Hydroxy-4-methylaminophenyl)carbamic acid 2-hydroxyethyl ester,
(2-Hydroxy-4-methylaminophenyl)carbamic acid isopropyl ester,
1-(2-Hydroxy-4-methylaminophenyl)urea,
3-(2-Hydroxy-4-methylaminophenyl)-1-methylurea,
3-(2-Hydroxy-4-methylaminophenyl)-1-ethylurea,
3-(2-Hydroxy-4-methylaminophenyl)1-(2-hydroxyethyl)urea,
3-(2-Hydroxy-4-methylaminophenyl)-1-isopropylurea,
3-(2-Hydroxy-4-methylaminophenyl)-1,1-dimethylurea,
3-(2-Hydroxy-4-methylaminophenyl)-1,1-diethylurea,
3-(2-Hydroxy-4-methylaminophenyl)-1,1-di(2-hydroxyethyl)urea,
(2-Hydroxy-4-(2-hydroxyethyl)aminophenyl)carbamic acid methyl ester,
(2-Hydroxy-4-(2-hydroxyethyl)aminophenyl)carbamic acid ethyl ester,
(2-Hydroxy-4-(2-hydroxyethyl)aminophenyl)carbamic acid 2-hydroxyethyl ester,
(2-Hydroxy-4-(2-hydroxyethyl)aminophenyl)carbamic acid isopropyl ester,
1-(2-Hydroxy-4-(2-hydroxyethyl)aminophenyl)urea,
3-(2-Hydroxy-4-(2-hydroxyethyl)aminophenyl)-1-methylurea,
3-(2-Hydroxy-4-(2-hydroxyethyl)aminophenyl)-1-ethylurea,
3-(2-Hydroxy-4-(2-hydroxyethyl)aminophenyl)-1-(2-hydroxyethyl)urea,
3-(2-Hydroxy-4-(2-hydroxyethyl)aminophenyl)-1-isopropylurea,
3-(2-Hydroxy-4-(2-hydroxyethyl)aminophenyl)-1,1-dimethylurea,
3-(2-Hydroxy-4-(2-hydroxyethyl)aminophenyl)-1,1-diethylurea,
3-(2-Hydroxy-4-(2-hydroxyethyl)aminophenyl)-1,1-di(2-hydroxyethyl)urea,
(2-Hydroxy-4-amino-5-chlorophenyl)carbamic acid methyl ester,
(2-Hydroxy-4-amino-5-chlorophenyl)carbamic acid ethyl ester,
(2-Hydroxy-4-amino-5-chlorophenyl)carbamic acid 2-hydroxyethyl ester,
(2-Hydroxy-4-amino-5-chlorophenyl)carbamic acid isopropyl ester,
1-(2-Hydroxy-4-amino-5-chlorophenyl)urea,
3-(2-Hydroxy-4-amino-5-chlorophenyl)-1-methylurea,
3-(2-Hydroxy-4-amino-5-chlorophenyl)-1-ethylurea,
3-(2-Hydroxy-4-amino-5-chlorophenyl)-1-(2-hydroxyethyl)urea,
3-(2-Hydroxy-4-amino-5-chlorophenyl)-1-isopropylurea,
3-(2-Hydroxy-4-amino-5-chlorophenyl)-1,1-dimethylurea,
3-(2-Hydroxy-4-amino-5-chlorophenyl)-1,1-diethylurea,
3-(2-Hydroxy-4-amino-5-chlorophenyl)-1,1-di(2-hydroxyethyl)urea,
(2-Hydroxy-4-methylamino-5-chlorophenyl)carbamic acid methyl ester,
(2-Hydroxy-4-methylamino-5-chlorophenyl)carbamic acid ethyl ester,
(2-Hydroxy-4-methylamino-5-chlorophenyl)carbamic acid 2-hydroxyethyl ester,
(2-Hydroxy-4-methylamino-5-chlorophenyl)carbamic acid isopropyl ester,
1-(2-Hydroxy-4-methylamino-5-chlorophenyl)urea,
3-(2-Hydroxy-4-methylamino-5-chlorophenyl)-1-methylurea,
3-(2-Hydroxy-4-methylamino-5-chlorophenyl)-1-ethylurea,
3-(2-Hydroxy-4-methylamino-5-chlorophenyl)-1-(2-hydroxyethyl)urea,
3-(2-Hydroxy-4-methylamino-5-chlorophenyl)-1-isopropylurea,
3-(2-Hydroxy-4-methylamino-5-chlorophenyl)-1,1-dimethylurea,
3-(2-Hydroxy-4-methylamino-5-chlorophenyl)-1,1-diethylurea,
3-(2-Hydroxy-4-methylamino-5-chlorophenyl)-1,1-di(2-hydroxyethyl)urea,
(2-Hydroxy-4-(2-hydroxyethyl)amino-5-chlorophenyl)carbamic acid methyl ester,
(2-Hydroxy-4-(2-hydroxyethyl)amino-5-chlorophenyl)carbamic acid ethyl ester,
(2-Hydroxy-4-(2-hydroxyethyl)amino-5-chlorophenyl)carbamic acid 2-hydroxyethyl ester,
(2-Hydroxy-4-(2-hydroxyethyl)amino-5-chlorophenyl)carbamic acid isopropyl ester,
1-(2-Hydroxy-4-(2-hydroxyethyl)amino-5-chlorophenyl)urea,
3-(2-Hydroxy-4-(2-hydroxyethyl)amino-5-chlorophenyl)-1-methylurea,
3-(2-Hydroxy-4-(2-hydroxyethyl)amino-5-chlorophenyl)-1-ethylurea,
3-(2-Hydroxy-4-(2-hydroxyethyl)amino-5-chlorophenyl)-1-(2-hydroxyethyl)urea,
3-(2-Hydroxy-4-(2-hydroxyethyl)amino-5-chlorophenyl)-1-isopropylurea,
3-(2-Hydroxy-4-(2-hydroxyethyl)amino-5-chlorophenyl)-1,1-dimethylurea,
3-(2-Hydroxy-4-(2-hydroxyethyl)amino-5-chlorophenyl)-1,1-diethylurea,
3-(2-Hydroxy-4-(2-hydroxyethyl)amino-5-chlorophenyl)-1,1-di(2-hydroxyethyl)urea,
(2-Hydroxy-4-amino-5-methoxyphenyl)carbamic acid methyl ester,
(2-Hydroxy-4-amino-5-methoxyphenyl)carbamic acid ethyl ester,
(2-Hydroxy-4-amino-5-methoxyphenyl)carbamic acid 2-hydroxyethyl ester,
(2-Hydroxy-4-amino-5-methoxyphenyl)carbamic acid isopropyl ester,
3-(2-Hydroxy-4-amino-5-methoxyphenyl)-1-(2-hydroxyethyl)urea,
3-(2-Hydroxy-4-amino-5-methoxyphenyl)-1,1-di(2-hydroxyethyl)urea,
(2-Hydroxy-4-methylamino-5-methoxyphenyl)carbamic acid methyl ester,
(2-Hydroxy-4-methylamino-5-methoxyphenyl)carbamic acid ethyl ester,
(2-Hydroxy-4-methylamino-5-methoxyphenyl)carbamic acid 2-hydroxyethyl ester,
(2-Hydroxy-4-methylamino-5-methoxyphenyl)carbamic acid isopropyl ester,
1-(2-Hydroxy-4-methylamino-5-methoxyphenyl)urea,
3-(2-Hydroxy-4-methylamino-5-methoxyphenyl)-1-methylurea,
3-(2-Hydroxy-4-methylamino-5-methoxyphenyl)-1-ethylurea,
3-(2-Hydroxy-4-methylamino-5-methoxyphenyl)-1-(2-hydroxyethyl)urea,
3-(2-Hydroxy-4-methylamino-5-methoxyphenyl)-1-isopropylurea,
3-(2-Hydroxy-4-methylamino-5-methoxyphenyl)-1,1-dimethylurea,
3-(2-Hydroxy-4-methylamino-5-methoxyphenyl)-1,1-diethylurea,
3-(2-Hydroxy-4-methylamino-5-methoxyphenyl)-1,1-di(2-hydroxyethyl)urea,
(2-Hydroxy-4-(2-hydroxyethyl)amino-5-methoxyphenyl)carbamic acid methyl ester,
(2-Hydroxy-4-(2-hydroxyethyl)amino-5-methoxyphenyl)carbamic acid ethyl ester,
(2-Hydroxy-4-(2-hydroxyethyl)amino-5-methoxyphenyl)carbamic acid 2-hydroxyethyl ester,
(2-Hydroxy-4-(2-hydroxyethyl)amino-5-methoxyphenyl)carbamic acid isopropyl ester,
1-(2-Hydroxy-4-(2-hydroxyethyl)amino-5-methoxyphenyl)urea,
3-(2-Hydroxy-4-(2-hydroxyethyl)amino-5-methoxyphenyl)-1-methylurea,
3-(2-Hydroxy-4-(2-hydroxyethyl)amino-5-methoxyphenyl)-1-ethylurea,
3-(2-Hydroxy-4-(2-hydroxyethyl)amino-5-methoxyphenyl)-1-(2-hydroxyethyl)urea,
3-(2-Hydroxy-4-(2-hydroxyethyl)amino-5-methoxyphenyl)-1-isopropylurea,
3-(2-Hydroxy-4-(2-hydroxyethyl)amino-5-methoxyphenyl)-1,1-dimethylurea,
3-(2-Hydroxy-4-(2-hydroxyethyl)amino-5-methoxyphenyl)-1,1-diethylurea,
3-(2-Hydroxy-4-(2-hydroxyethyl)amino-5-methoxyphenyl)-1,1-di(2-hydroxyethyl)urea,
(2-Hydroxy-3-methoxycarbonylaminophenyl)carbamic acid methyl ester,
(2-Hydroxy-3-ethoxycarbonylaminophenyl)carbamic acid ethyl ester,
(2-Hydroxy-3-(2-hydroxyethoxy)carbonylaminophenyl)-carbamic acid 2-hydroxyethyl ester,
(2-Hydroxy-3-isopropoxycarbonylaminophenyl)carbamic acid isopropyl ester,
1-(2-Hydroxy-3-ureidophenyl)urea,
3-(2-Hydroxy-3-(3-methylureido)phenyl)-1-methylurea,
3-(2-Hydroxy-3-(3-ethylureido)phenyl)-1-ethylurea,
3-(2-Hydroxy-3-(3-(2-hydroxyethyl)ureido)phenyl)-1-(2-hydroxyethyl)urea,
3-(2-Hydroxy-3-(3-isopropylureido)phenyl)-1-isopropylurea,
3-(2-Hydroxy-3-(3,3-dimethylureido)phenyl)-1,1-dimethylurea,
and their addition salts with an acid.

19. Composition according to claim 18, characterized in that the compounds of formula (I) are chosen from:
(2-Hydroxy-4-aminophenyl)carbamic acid methyl ester,
(2-Hydroxy-4-aminophenyl)carbamic acid ethyl ester,
(2-Hydroxy-4-aminophenyl)carbamic acid 2-hydroxyethyl ester,
(2-Hydroxy-4-aminophenyl)carbamic acid isopropyl ester,
1-(2-Hydroxy-4-aminophenyl)urea,
3-(2-Hydroxy-4-aminophenyl)-1-methylurea,
3-(2-Hydroxy-4-aminophenyl)-1-ethylurea,
3-(2-Hydroxy-4-aminophenyl)-1-(2-hydroxyethyl)urea,
3-(2-Hydroxy-4-aminophenyl)-1-isopropylurea,
3-(2-Hydroxy-4-aminophenyl)-1,1-dimethylurea,
(2-Hydroxy-4-methylaminophenyl)carbamic acid methyl ester,
(2-Hydroxy-4-methylaminophenyl)carbamic acid ethyl ester,
(2-Hydroxy-4-methylaminophenyl)carbamic acid 2-hydroxyethyl ester,
(2-Hydroxy-4-methylaminophenyl)carbamic acid isopropyl ester,
1-(2-Hydroxy-4-methylaminophenyl)urea,
3-(2-Hydroxy-4-methylaminophenyl)-1-methylurea,
3-(2-Hydroxy-4-methylaminophenyl)-1-ethylurea,
3-(2-Hydroxy-4-methylaminophenyl)-1-(2-hydroxyethyl)urea,
3-(2-Hydroxy-4-methylaminophenyl)-1-isopropylurea,
3-(2-Hydroxy-4-methylaminophenyl)-1,1-dimethylurea,
(2-Hydroxy-4-(2-hydroxyethyl)aminophenyl)carbamic acid methyl ester,
(2-Hydroxy-4-(2-hydroxyethyl)aminophenyl)carbamic acid ethyl ester,
(2-Hydroxy-4-(2-hydroxyethyl)aminophenyl)carbamic acid 2-hydroxyethyl ester,
(2-Hydroxy-4-(2-hydroxyethyl)aminophenyl)carbamic acid isopropyl ester,
1-(2-Hydroxy-4-(2-hydroxyethyl)aminophenyl)urea,
3-(2-Hydroxy-4-(2-hydroxyethyl)aminophenyl)-1-methylurea,
3-(2-Hydroxy-4-(2-hydroxyethyl)aminophenyl)-1-ethylurea,
3-(2-Hydroxy-4-(2-hydroxyethyl)aminophenyl)-1-(2-hydroxyethyl)urea,
3-(2-Hydroxy-4-(2-hydroxyethyl)aminophenyl)-1-isopropylurea,
3-(2-Hydroxy-4-(2-hydroxyethyl)aminophenyl)-1,1-dimethylurea,
(2-Hydroxy-4-amino-5-chlorophenyl)carbamic acid methyl ester,
(2-Hydroxy-4-amino-5-chlorophenyl)carbamic acid ethyl ester,
(2-Hydroxy-4-amino-5-chlorophenyl)carbamic acid 2-hydroxyethyl ester,
(2-Hydroxy-4-amino-5-chlorophenyl)carbamic acid isopropyl ester,
1-(2-Hydroxy-4-amino-5-chlorophenyl)urea,
3-(2-Hydroxy-4-amino-5-chlorophenyl)-1-methylurea,
3-(2-Hydroxy-4-amino-5-chlorophenyl)-1-ethylurea,
3-(2-Hydroxy-4-amino-5-chlorophenyl)-1-(2-hydroxyethyl)urea,
3-(2-Hydroxy-4-amino-5-chlorophenyl)-1-isopropylurea,
3-(2-Hydroxy-4-amino-5-chlorophenyl)-1,1-dimethylurea,
(2-Hydroxy-4-methylamino-5-chlorophenyl)carbamic acid methyl ester,
(2-Hydroxy-4-methylamino-5-chlorophenyl)carbamic acid ethyl ester,
(2-Hydroxy-4-methylamino-5-chlorophenyl)carbamic acid 2-hydroxyethyl ester,
(2-Hydroxy-4-methylamino-5-chlorophenyl)carbamic acid isopropyl ester,
1-(2-Hydroxy-4-methylamino-5-chlorophenyl)urea,
3-(2-Hydroxy-4-methylamino-5-chlorophenyl)-1-methylurea,
3-(2-Hydroxy-4-methylamino-5-chlorophenyl)-1-ethylurea,
3-(2-Hydroxy-4-methylamino-5-chlorophenyl)-1-(2-hydroxyethyl)urea,
3-(2-Hydroxy-4-methylamino-5-chlorophenyl)-1-isopropylurea,
3-(2-Hydroxy-4-methylamino-5-chlorophenyl)-1,1-dimethylurea,
(2-Hydroxy-4-(2-hydroxyethyl)amino-5-chlorophenyl)carbamic acid methyl ester,
(2-Hydroxy-4-(2-hydroxyethyl)amino-5-chlorophenyl)carbamic acid ethyl ester,
(2-Hydroxy-4-(2-hydroxyethyl)amino-5-chlorophenyl)carbamic acid 2-hydroxyethyl ester,
(2-Hydroxy-4-(2-hydroxyethyl)amino-5-chlorophenyl)carbamic acid isopropyl ester,
1-(2-Hydroxy-4-(2-hydroxyethyl)amino-5-chlorophenyl)urea,
3-(2-Hydroxy-4-(2-hydroxyethyl)amino-5-chlorophenyl)-1-methylurea,
3-(2-Hydroxy-4-(2-hydroxyethyl)amino-5-chlorophenyl)-1-ethylurea,
3-(2-Hydroxy-4-(2-hydroxyethyl)amino-5-chlorophenyl)-1-(2-hydroxyethyl)urea,
3-(2-Hydroxy-4-(2-hydroxyethyl)amino-5-chlorophenyl)-1-isopropylurea,
3-(2-Hydroxy-4-(2-hydroxyethyl)amino-5-chlorophenyl)-1,1-dimethylurea,
(2-Hydroxy-4-amino-5-methoxyphenyl)carbamic acid methyl ester,
(2-Hydroxy-4-amino-5-methoxyphenyl)carbamic acid ethyl ester,
(2-Hydroxy-4-amino-5-methoxyphenyl)carbamic acid 2-hydroxyethyl ester,
(2-Hydroxy-4-amino-5-methoxyphenyl)carbamic acid isopropyl ester,
3-(2-Hydroxy-4-amino-5-methoxyphenyl)-1-(2-hydroxyethyl)urea,
(2-Hydroxy-4-methylamino-5-methoxyphenyl)carbamic acid methyl ester,
(2-Hydroxy-4-methylamino-5-methoxyphenyl)carbamic acid ethyl ester,
(2-Hydroxy-4-methylamino-5-methoxyphenyl)carbamic acid 2-hydroxyethyl ester,
(2-Hydroxy-4-methylamino-5-methoxyphenyl)carbamic acid isopropyl ester,
1-(2-Hydroxy-4-methylamino-5-methoxyphenyl)urea,
3-(2-Hydroxy-4-methylamino-5-methoxyphenyl)-1-methylurea,
3-(2-Hydroxy-4-methylamino-5-methoxyphenyl)-1-ethylurea,
3-(2-Hydroxy-4-methylamino-5-methoxyphenyl)-1-(2-hydroxyethyl)urea,
3-(2-Hydroxy-4-methylamino-5-methoxyphenyl)-1-isopropylurea,
3-(2-Hydroxy-4-methylamino-5-methoxyphenyl)-1,1-dimethylurea,
(2-Hydroxy-4-(2-hydroxyethyl)amino-5-methoxyphenyl)carbamic acid methyl ester,
(2-Hydroxy-4-(2-hydroxyethyl)amino-5-methoxyphenyl)carbamic acid ethyl ester,
(2-Hydroxy-4-(2-hydroxyethyl)amino-5-methoxyphenyl)carbamic acid 2-hydroxyethyl ester,
(2-Hydroxy-4-(2-hydroxyethyl)amino-5-methoxyphenyl)carbamic acid isopropyl ester,
1-(2-Hydroxy-4-(2-hydroxyethyl)amino-5-methoxyphenyl)urea,
3-(2-Hydroxy-4-(2-hydroxyethyl)amino-5-methoxyphenyl)-1-methylurea,
3-(2-Hydroxy-4-(2-hydroxyethyl)amino-5-methoxyphenyl)-1-ethylurea,
3-(2-Hydroxy-4-(2-hydroxyethyl)amino-5-methoxyphenyl)-1-(2-hydroxyethyl)urea,
3-(2-Hydroxy-4-(2-hydroxyethyl)amino-5-methoxyphenyl)-1-isopropylurea,
3-(2-Hydroxy-4-(2-hydroxyethyl)amino-5-methoxyphenyl)-1,1-dimethylurea, and their addition salts with an acid.

20. Composition according to claim 19, characterized in that the compounds of formula (I) are chosen from:
(2-Hydroxy-4-aminophenyl)carbamic acid methyl ester,
(2-Hydroxy-4-aminophenyl)carbamic acid ethyl ester,
(2-Hydroxy-4-aminophenyl)carbamic acid 2-hydroxyethyl ester,
(2-Hydroxy-4-aminophenyl)carbamic acid isopropyl ester,
1-(2-Hydroxy-4-aminophenyl)urea,
3-(2-Hydroxy-4-aminophenyl)-1-methylurea,
3-(2-Hydroxy-4-aminophenyl)-1-ethylurea,
3-(2-Hydroxy-4-aminophenyl)-1-(2-hydroxyethyl)urea,
3-(2-Hydroxy-4-aminophenyl)-1-isopropylurea,
3-(2-Hydroxy-4-aminophenyl)-1,1-dimethylurea,
(2-Hydroxy-4-amino-5-chlorophenyl)carbamic acid methyl ester,
(2-Hydroxy-4-amino-5-chlorophenyl)carbamic acid ethyl ester,
(2-Hydroxy-4-amino-5-chlorophenyl)carbamic acid 2-hydroxyethyl ester,
(2-Hydroxy-4-amino-5-chlorophenyl)carbamic acid isopropyl ester,
1-(2-Hydroxy-4-amino-5-chlorophenyl)urea,
3-(2-Hydroxy-4-amino-5-chlorophenyl)-1-methylurea,
3-(2-Hydroxy-4-amino-5-chlorophenyl)-1-ethylurea,
3-(2-Hydroxy-4-amino-5-chlorophenyl)-1-(2-hydroxyethyl)urea,
3-(2-Hydroxy-4-amino-5-chlorophenyl)-1-isopropylurea,
3-(2-Hydroxy-4-amino-5-chlorophenyl)-1,1-dimethylurea,
(2-Hydroxy-4-amino-5-methoxyphenyl)carbamic acid methyl ester,
(2-Hydroxy-4-amino-5-methoxyphenyl)carbamic acid ethyl ester,
(2-Hydroxy-4-amino-5-methoxyphenyl)carbamic acid 2-hydroxyethyl ester,
(2-Hydroxy-4-amino-5-methoxyphenyl)carbamic acid isopropyl ester,
3-(2-Hydroxy-4-amino-5-methoxyphenyl)-1-(2-hydroxyethyl)urea, and their addition salts with an acid.

21. Composition according to any one of the preceding claims, characterized in that the compound(s) of formula (I) and/or its (their) addition salts with an acid preferably represent(s) from 0.0005 to 12% by weight of the total weight of the dyeing composition.

22. Composition according to any one of the preceding claims, characterized in that it contains at least the ethyl ester of (2-hydroxy-4-aminophenyl)-carbamic acid or one of its addition salts with an acid as compound of formula (I) in combination with at least one pyrazolopyrimidine chosen from 5-methylpyrazolo[1,5-a]pyrimidine-3,7-diamine; pyrazolo[1,5-a]pyrimidine-3,7-diamine; 2-methylpyrazolo[1,5-a]pyrimidine-3,7-diamine; 3-amino-5-methyl-7-imidazolylpropylaminopyrazolo[1,5-a]-pyrimidine; 3-amino-5,6-dimethyl-7-imidazolylpropylaminopyrazolo[1,5-a]pyrimidine; 2-methoxy-5,7-dimethylpyrazolo[1,5-a]pyrimidin-3-ylamine; 3-amino-7-imidazolylpropylaminopyrazolo[1,5-a]-pyrimidine; and their addition salts with an acid.

23. Composition according to claim 22, characterized in that it contains the combination of 5-methylpyrazolo[1,5-a]pyrimidine-3,7-diamine or of one of its addition salts with an acid and the ethyl ester of (2-hydroxy-4-aminophenyl)carbamic acid or one of its addition salts with an acid.

24. Composition according to any one of the preceding claims, characterized in that the addition salts with an acid are chosen from hydrochlorides, hydrobromides, sulphates, citrates, succinates, tartrates, lactates and acetates.

25. Method of dyeing keratinous fibres, and in particular human keratinous fibres such as hair, characterized in that at least one dyeing composition as defined in any one of claims 1 to 24 is applied to these fibres, and in that the colour is developed at acidic, neutral or alkaline pH with the aid of an oxidizing agent which is added just at the time of use to the dyeing composition or which is present in an oxidizing composition applied simultaneously or sequentially in a separate manner.

26. Method according to claim 25, characterized in that the oxidizing agent is chosen from hydrogen peroxide, urea peroxide, alkali metal bromates, persalts and enzymes.

27. Method according to claim 26, characterized in that the enzymes are chosen from peroxidases, laccases, tyrosinases and oxidoreductases.

28. Multicompartment device, or multicompartment dyeing <<kit>>, in which a first compartment contains a dyeing composition as defined in any one of claims 1 to 24 and a second compartment contains an oxidizing composition.
